SEQUENCE LISTING (19 sequences)

SEQ ID NO: 1 - DNA, 27 bp, synthetic construct
  misc_feature 1..27: note = Synthesized in the lab
  source 1..27: mol_type = other DNA
cagactaccatgacctgagtcbbbbbb

SEQ ID NO: 2 - DNA, 38 bp, synthetic construct
  misc_feature 1..38: note = Synthesized in the lab
  misc_difference 38: note = where N represents any of A, T, C or G
  source 1..38: mol_type = other DNA
cagactaccatgacctgagtctttttttttttttttvn

SEQ ID NO: 3 - DNA, 29 bp, synthetic construct
  misc_feature 1..29: note = Synthesized in the lab
  source 1..29: mol_type = other DNA
gtcagagtcgaatgcgtactgbbbbbbbb

SEQ ID NO: 4 - DNA, 21 bp, synthetic construct
  misc_feature 1..21: note = Synthesized in the lab
  source 1..21: mol_type = other DNA
  modified_base 9: mod_base = OTHER; note = Methylated nucleotide
cagactaccatgacctgagtc

SEQ ID NO: 5 - DNA, 21 bp, synthetic construct
  misc_feature 1..21: note = Synthesized in the lab
  source 1..21: mol_type = other DNA
  modified_base 9: mod_base = OTHER; note = Methylated nucleotide
gtcagagtcgaatgcgtactg

SEQ ID NO: 6 - DNA, 19 bp, synthetic construct
  misc_feature 1..19: note = Artificial Sequence
  source 1..19: mol_type = other DNA
agatgtgtataagagacag

SEQ ID NO: 7 - DNA, 23 bp, synthetic construct
  misc_feature 1..23: note = Synthesized in the lab
  misc_difference 1..4: note = where N represents any of A, T, C or G
  source 1..23: mol_type = other DNA
nnnnctgtctcttatacacatct

SEQ ID NO: 8 - DNA, 33 bp, synthetic construct
  misc_feature 1..33: note = Synthesized in the lab
  source 1..33: mol_type = other DNA
tcgtcggcagcgtcagatgtgtataagagacag

SEQ ID NO: 9 - DNA, 34 bp, synthetic construct
  misc_feature 1..34: note = Synthesized in the lab
  source 1..34: mol_type = other DNA
gtctcgtgggctcggagatgtgtataagagacag

SEQ ID NO: 10 - DNA, 27 bp, synthetic construct
  misc_feature 1..27: note = Synthesized in the lab
  misc_difference 22..27: note = where N represents any of A, T, C or G
  source 1..27: mol_type = other DNA
cagactaccatgacctgagtcnnnnnn

SEQ ID NO: 11 - DNA, 27 bp, synthetic construct
  misc_feature 1..27: note = Synthesized in the lab
  source 1..27: mol_type = other DNA
cagactaccatgacctgagtcdddddd

SEQ ID NO: 12 - DNA, 27 bp, synthetic construct
  misc_feature 1..27: note = Synthesized in the lab
  source 1..27: mol_type = other DNA
cagactaccatgacctgagtchhhhhh

SEQ ID NO: 13 - DNA, 29 bp, synthetic construct
  misc_feature 1..29: note = Synthesized in the lab
  misc_difference 22..29: note = where N represents any of A, T, C or G
  source 1..29: mol_type = other DNA
gtcagagtcgaatgcgtactgnnnnnnnn

SEQ ID NO: 14 - DNA, 29 bp, synthetic construct
  misc_feature 1..29: note = Synthesized in the lab
  source 1..29: mol_type = other DNA
gtcagagtcgaatgcgtactgdddddddd

SEQ ID NO: 15 - DNA, 29 bp, synthetic construct
  misc_feature 1..29: note = Synthesized in the lab
  source 1..29: mol_type = other DNA
gtcagagtcgaatgcgtactghhhhhhhh

SEQ ID NO: 16 - DNA, 21 bp, synthetic construct
  source 1..21: mol_type = other DNA
gtcagagtcgaatgcgtactg

SEQ ID NO: 17 - DNA, 21 bp, synthetic construct
  source 1..21: mol_type = other DNA
cagactaccatgacctgagtc

SEQ ID NO: 18 - DNA, 12 bp, synthetic construct
  source 1..12: mol_type = other DNA
  misc_difference 12: note = n represents any of A, T, C or G
ttttttttttvn

SEQ ID NO: 19 - DNA, 11 bp, synthetic construct
  source 1..11: mol_type = other DNA
  misc_difference 11: note = n represents any of A, T, C or G
tttttttttvn

(19) United States
(12) Patent Application Publication
(10) Pub. No.: US 2025/0346887 A1
(43) Pub. Date: Nov. 13, 2025

(54) RAPID PATHOGEN IDENTIFICATION AND DETECTION MOLECULAR DIAGNOSTICS TECHNOLOGY

(71) Applicant: AGENCY FOR SCIENCE, TECHNOLOGY AND RESEARCH (A*STAR), SINGAPORE (SG)

(72) Inventors: LIAN CHYE WINSTON KOH, SINGAPORE (SG); YIQI SEOW, SINGAPORE (SG); SIANG SHAWN HOON, SINGAPORE (SG)

(21) Appl. No.: 18/727,017

(22) PCT Filed: Jan. 7, 2023
(86) PCT No.: PCT/IB2023/050130

§ 371 Date: Jul. 5, 2024

Related U.S. Application Data
(60) Provisional application No. 63/297,241, filed on Jan. 7, 2022.
(60) Provisional application No. 63/406,745, filed on Sep. 15, 2022.

(51) Int. Cl. C12N 15/10

(52) U.S. Cl. CPC C12N 15/1093

(57) ABSTRACT

The present invention provides a method of detecting non-host species in a host sample. The host sample is contacted with magnetic particles coupled to enzymes to degrade cell-free host nucleic acids present in the host sample, wherein the magnetic particles coupled to enzymes are capable of degrading both DNA and RNA. A magnetic field is applied to remove the magnetic particles coupled to enzymes from the host sample. A DNA and RNA library is created from the degraded sample in a one-pot process, followed by detection of the presence of non-host nucleic acids from the DNA and RNA library.

### CROSS-REFERENCE TO RELATED APPLICATIONS

The present application claims the priority benefit of (1) U.S. provisional patent application No. 63/297,241, filed Jan. 7, 2022, and (2) U.S. provisional patent application No. 63/406,745, filed Sep. 15, 2022, the contents of which are incorporated herein in their entireties by reference thereto.

### SEQUENCE LISTING

The instant application contains a Sequence Listing which has been submitted electronically in XML format and is hereby incorporated by reference in its entirety. Said XML copy, created on Feb. 6, 2025, is named 245866_000013_SL.xml and is 27,194 bytes in size.

### TECHNICAL FIELD

The present disclosure relates broadly to methods for detecting non-host species in host samples including techniques for depletion of host background to enrich pathogen DNA and RNA and one-pot DNA/RNA library preparation.

### BACKGROUND

The ability to detect non-host species residing within or on a host is important for microbiological research, for pathogen identification and for clinical diagnosis in human, pets, livestock and wild animals. High throughput sequencing can be a powerful method for detecting non-host nucleic acids in host fluids, but the nucleic acid signal from non-host organisms is frequently swamped out by signal from the host.

Taking mammalian bodily fluids, such as plasma, cerebrospinal fluid, nasal excretion and urine, as typical examples, even when host cells are depleted by low speed centrifugation, a significant amount of mammalian nucleic acids can still be found in a host of complexes, including lipoprotein complexes, extracellular vesicles, mitochondria and apoptotic bodies. Depletion of these host signals is crucial to getting a good signal-to-background ratio for high throughput sequencing.

A number of methods have been used in the art to remove such background host signals. Several of such methods involve the further use of high-speed centrifugation followed by selective cell lysis and nucleic acid degradation. However, nucleic acid signal from non-host organisms including bacteria, protozoa, fungi, and DNA/RNA viruses, may be lost during the centrifugation step. Other drawbacks include limitations on the specific types of host cells nucleic acids that may be degraded or inhibited and insufficient enrichment of non-host organisms' signals.

Following removal of background host signals, metagenomics sequencing involves creation of a library. Typically, separate samples are used to separately create a DNA library and an RNA library. Preparation of libraries for high throughput sequencing from biological or medical samples is not always easy because they are usually significantly low in the amount of nucleic acids. Many conventional methods, in order to cope with this limitation, may require more complex procedure which is more time-consuming and costly, or limited to certain kind of nucleic acid sample, or may lose or add some elements that do not belong to the original sample, implying that there is no simultaneous amplification of the nucleic acid sample in place which avoids or lowers bias between different types of nucleic acids for the same target, especially when the amount of the nucleic acid sample is only up to picogram level. Therefore, separate creation of DNA and RNA libraries can be difficult, increase the cost of processing, and increase the sample processing time.

U.S. Pat. No. 5,731,171 (Bohlander et al.) provided a sequence-independent amplification (SIA), PCR-based method capable of amplifying DNA from minute amounts such as from microdissected chromosomal material. The method involves an initial primer composed of 4-8 random nucleotides, at 3′ end and 10-30 nucleotides of defined (non-random) sequence at 5′ end, where the random nucleotide can be any of G/A/T/C (in any order). The 3′ end of the initial primer should be complementary to random sites throughout the target DNA segments while the defined sequence should constitute a PCR primer which do not form self-homologies, no runs of the same nucleotide, and no overly rich G:C or A:T rich.

U.S. Pat. No. 6,124,120 (Lizardi et al.) provided a non-PCR-based method of multiple strand displacement amplification (MDA) using two set of primers complementary to a pair of double-stranded DNA, where some intervening primers are displaced during replication by the polymerase. Another embodiment of this patent also uses a random set of primers to sequence whole genome. A highly processive polymerase is used in the replication such that overlapping copies of the entire genome can be synthesized in a short time.

However, both Bohlander et al. and Lizardi et al. could not be used to amplify samples with a mix of different nucleic acids including deoxyribonucleic acid (DNA), ribonucleic acid (RNA), or any analogues thereof, which is/are either single-stranded or double-stranded, or both, simultaneously without bias.

U.S. Pat. No. 7,402,386 (Kum et al.) disclosed using a RNA-DNA composite primer and DNA- and RNA-dependent DNA polymerases for globally amplifying DNA/RNA polynucleotide targets. The method involved cleavage of RNA portion from RNA/DNA heteroduplex before subsequent amplification.

U.S. Pat. Nos. 7,718,403 and 8,206,913 (Kamberov et al.) disclosed methods for whole genome amplification (WGA) and whole transcriptome amplification (WTA) including a library generation step and a library amplification step, which involve the use of random primer and specific DNA polymerase in the library generation step to generate the first strand from the DNA/RNA template, where the variable region of the primer comprises at most 3 random nucleotides each composed of two non-complementary nucleotides such that primers will not self-hybridize or cross-hybridize with each other. Choosing random nucleotides composed of two non-complementary nucleotide bases instead of random nucleotides composed of at least three non-complementary nucleotide bases does not effectively reduce cross-hybridization among the pool of random primers if there are only at most 3 random nucleotides in each of the random primer because PCR favors generation of short amplicons. It is important to reduce the ability to form non-specific amplicons in order to prevent sequestration of polymerase towards unproductive amplicons.

U.S. Pat. No. 8,741,606 (Casbon et al.) disclosed a method of tagging degenerate base region (DBR) to a nucleic acid molecule on both ends to be sequenced to result in an asymmetrically tagged nucleic acid molecule because two different DBRs. The DBRs include a sequencing primer site for subsequent PCR. In certain embodiments of '606, DBR may be 3 to 10 random nucleotides-long, and each DBR may have different base composition such as 4-base DBR may have any of the following compositions: NNNN; NRSN; SWSW; BDHV (according to IUPAC nucleotide code). '606 involves addition of two different DBRs on both ends of the nucleic acid molecule to be sequenced, and some functional domains such as sequencing primer site and unique multiplex identifier for sequencing purpose, but it was not primarily designed for library preparation from a mix of different nucleic acids including both DNA and RNA in either single-stranded or double-stranded form.

U.S. Pat. No. 8,728,766 (Casbon et al.) disclosed a method for processing a genomic DNA sample including using a population of first primers to hybridize a genomic sample of initial target DNA molecules, where the first primers include different DBR sequences 5′ to a target-specific sequence, and different DBR sequences include at least one of R, Y, S, W, K, M, B, D, H, V, N according to IUPAC nucleotide code, or their modified versions. In certain examples, RYB serves as DBR sequence while DHVB serves as target-specific sequence. In those example, the total number of different sequences from those random nucleotides could be 972 (2×2×3×3×3×3×3). There were still only three random nucleotides in the DBR sequence employed in the first primers of '766. Cross-hybridization among primers could still happen under suitable conditions.

U.S. Pat. No. 9,920,355 (Osborne et al.) provided a method of library preparation comprising including deoxy-methyl-cytidine triphosphate in different concentrations in the RT reaction mix for first strand, second strand generations, and/or PCR amplifications to facilitate fragmentation using a specific restriction enzyme digestion.

In view of the above, there is a need to address or at least ameliorate one or more of the above-mentioned problems. In particular, there is a need to provide a method of degrading unwanted nucleic acids in a sample along with a need to rapidly create DNA and RNA libraries in a cost-effective manner so that detecting non-host species in a host sample and associated compositions can be accurately and rapidly performed. Such techniques enable widespread use of next generation metagenomic sequencing, particularly for pathogen detection in humans.

### SUMMARY OF THE INVENTION

In one aspect, the present invention provides an improved metagenomics sequencing process of a body fluid sample. In one part of the process unwanted nucleic acids are degraded in the sample by contacting the sample with magnetic particles coupled to enzymes such as nucleases to degrade unwanted nucleic acids present in the sample. The magnetic particles are coupled to enzymes and are capable of degrading both DNA and RNA followed by applying a magnetic field to remove the magnetic particles coupled to enzymes from the sample. A DNA and RNA library is created from the degraded sample in a one-pot process.

In a further aspect, the one-pot process uses a set of primers to prime single-stranded DNA and at the same time prime RNA.

In a further aspect, the sample includes more than one type of nucleic acid including single-stranded and/or double-stranded DNA and/or RNA as a template of subsequent extensions and amplifications and the one-pot process includes preparing a first DNA strand from the sample including annealing one or more first DNA strand generation primers to any of the DNA and/or RNA template, and extending from the annealed first DNA strand generation primer including employing a DNA polymerase that enables synthesis of the first DNA strand from either or both of DNA and/or RNA templates to obtain the first DNA strand.

In a further aspect, the magnetic particles coupled to enzymes include magnetic particles coupled to both a DNAse and a RNAse, a magnetic particle coupled to an enzyme capable of degrading both DNA and RNA, a mixture comprising a magnetic particle coupled to a DNAse and a magnetic particle coupled to a RNAse, or combinations thereof.

In a further aspect, the unwanted nucleic acids comprise cell-free nucleic acids.

In a further aspect, degrading unwanted nucleic acids in the sample further includes applying a lysing agent to the sample for selectively lysing biological complexes in the sample to free said unwanted nucleic acids for degradation by the enzymes, optionally wherein the lysing agent is substantially incapable of lysing viruses and intact cells selected from bacteria, protozoa, fungi and parasites.

In a further aspect, the lysing agent comprise a mild detergent.

In a further aspect, the sample is a sample derived from a host subject and the unwanted nucleic acids are host nucleic acids but are substantially devoid of non-host nucleic acids.

In another aspect, the present invention provides a method of detecting non-host species in a host sample. The host sample is contacted with magnetic particles coupled to enzymes to degrade cell-free host nucleic acids present in the host sample, wherein the magnetic particles coupled to enzymes are capable of degrading both DNA and RNA. A magnetic field is applied to remove the magnetic particles coupled to enzymes from the host sample. A DNA and RNA library is created from the degraded sample in a one-pot process, followed by detection of the presence of non-host nucleic acids from the DNA and RNA library.

In a further aspect, the one-pot process uses a set of primers to prime single-stranded DNA and at the same time prime RNA.

In a further aspect, the sample includes more than one type of nucleic acid including single-stranded and/or double-stranded DNA and/or RNA as a template of subsequent extensions and amplifications and the one-pot process includes preparing a first DNA strand from the sample including annealing one or more first DNA strand generation primers to any of the DNA and/or RNA template, and extending from the annealed first DNA strand generation primer including employing a DNA polymerase that enables synthesis of the first DNA strand from either or both of DNA and/or RNA templates to obtain the first DNA strand.

In a further aspect, host cells are removed from the host sample prior to contacting the host sample with magnetic particles coupled to enzymes.

In a further aspect, a first lysing agent is applied to the host sample for selectively lysing biological complexes to free host nucleic acids contained therein prior to the contacting step, optionally wherein the first lysing agent is substantially incapable of lysing non-host species to free the nucleic acids contained therein for degradation by the enzymes. The lysing agent comprise a mild detergent.

The step of detecting the presence of non-host nucleic acids in the sample includes applying a second lysing agent to the host sample for lysing non-host species to free the nucleic acids contained therein.

In a further aspect, the second lysing agent comprises lysing particles that are capable of imparting a mechanical force to lyse the non-host species and free the nucleic acids contained therein.

In a further aspect, detecting the presence of non-host nucleic acids in the sample includes sequencing the non-host nucleic acids.

### BRIEF DESCRIPTION OF THE DRAWINGS

FIG. 1 is a schematic flow diagram showing various process steps in accordance with an exemplary embodiment of the method disclosed herein.

FIG. 2 shows amplification plots showing the amplification curve of PCR products using primers specific for each non-host organism or representative human genes in accordance with an exemplary embodiment disclosed herein.

FIG. 3 schematically illustrates an embodiment of the present method to generate double-stranded DNA molecules from different nucleic acids using the first strand generation primers, second strand generation primers and corresponding amplification primers of the present invention. Figure discloses “TTTTTTTTTVN” as SEQ ID NO: 19.

FIG. 4 schematically illustrates an embodiment of the present method to modify and fragment the double-stranded DNA molecules obtained from amplification of the second DNA strand as shown in FIG. 3 to generate double-strand DNA molecules with a linear length of shorter than 1000 nucleotides.

FIG. 5 schematically illustrates an embodiment of the present method to append corresponding adaptor sequences to the fragmented double-stranded DNA molecules as shown in FIG. 4 for barcoding. Figure discloses SEQ ID NO: 19.

FIG. 6 schematically illustrates an embodiment of the present invention to prepare library from a sample with RNA template according to the present method and components described herein. Figure discloses “TTTTTTTTTVN” as SEQ ID NO: 19.

FIGS. 7A-7D illustrate the results of normalization of input RNA by qPCR using primers against human 18S (FIG. 7A), U2AF1 (FIG. 7B), GAPDH coding region (FIG. 7C) and GAPDH 3′UTR (FIG. 7D) from 5 ng/500 pg HEK293 cell RNA (dT 5 ng/500 pg) and the cleaned library preparation prepared using 5 ng/500 pg dT-adaptor B6 sequence (dT:B6 5 g/500 pg) according to Example 3.

FIG. 8 illustrates two plots of the transcript reads between the samples using 5 ng of dT-adaptor B6 sequence (dT:B6 5 ng) and 5 ng of dT, and between dT:B6 5 ng and dT:B6 500 pg.

### DEFINITIONS

The term “nucleic acids” as used herein broadly encompasses ribonucleic acids (RNA), deoxyribonucleic acid (DNA) or parts/fragments thereof. The term also encompasses all forms of nucleic acids including intracellular nucleic acids, extracellular nucleic acids, acellular nucleic acids, unencapsulated nucleic acids, encapsulated nucleic acids, protected nucleic acids, unprotected nucleic acids, cell-free nucleic acids, human/animal/mammalian nucleic acids, microorganism nucleic acids (e.g., bacteria, fungi, virus), host cell nucleic acids and non-host cell nucleic acids.

The term “unwanted nucleic acids” as used herein refers to nucleic acids that are not desired to be present as intact nucleic acids for a particular step but it does not necessarily mean that these nucleic acids have entirely no use or purpose such as for other steps or methods. For example, if a particular step requires a sample to be free from cell-free host nucleic acids, these cell-free host nucleic acids may be deemed to be unwanted for that particular step even though such nucleic acids may be useful for other steps. Accordingly, cell-free host nucleic acids may be deemed unwanted for a particular step and therefore removed/degraded from a sample prior to or at the particular step; and when they are no longer unwanted or become desirable to be present in other steps, they may be reintroduced into the sample e.g., by lysing intact host cells.

The term “magnetic” as used herein refers to magnetic properties of a material. The term “magnetic particles” as used herein refers to particles that are made from materials that possess magnetic properties. Magnetic particles are capable of interacting with a magnetic field, generating either an attractive force or a repulsive force. If a magnetic field is applied to a magnetic particle, the particle becomes magnetized. The magnetized particle may be classified into ferromagnetic or paramagnetic, depending on the type of magnetization resulting from the application of a magnetic field. A ferromagnetic material is a substance which is strongly magnetized in the same direction as a magnetic field when a strong magnetic field is externally applied and remains magnetized even after the external magnetic field is removed. Magnetic particles may have average diameters in the range of nanometers, micrometers, or millimeters. Magnetic particles may include magnetic beads/microbeads and/or nanoparticles.

The term “micro” as used herein is to be interpreted broadly to include dimensions from about 1 micron to about 1000 microns.

The term “nano” as used herein is to be interpreted broadly to include dimensions less than about 1000 nm.

The term “particle” as used herein broadly refers to a discrete entity or a discrete body. The particle described herein can include an organic, an inorganic or a biological particle. The particle used described herein may also be a macro-particle that is formed by an aggregate of a plurality of sub-particles or a fragment of a small object. The particle of the present disclosure may be spherical, substantially spherical, or non-spherical, such as irregularly shaped particles or ellipsoidally shaped particles. The term “size” when used to refer to the particle broadly refers to the largest dimension of the particle. For example, when the particle is substantially spherical, the term “size” can refer to the diameter of the particle; or when the particle is substantially non-spherical, the term “size” can refer to the largest length of the particle.

“One-pot” described herein may refer to a single step of using a set of primers to prime single-stranded DNA and at the same time prime RNA such as mRNA (e.g., by a primer with oligo-dT sequence at the 5′ end of the two random nucleotides), with a polymerase with both DNA extension and RNA transcription abilities under suitable reverse transcription conditions so it can generate cDNA strand from both DNA and RNA templates, or any other oligonucleotide synthesis without additional purification and/or other methods to isolate one type of nucleic acid from the others because of the limitation of the oligonucleotide synthesis scheme provided by conventional technologies.

The terms “coupled” or “connected” as used in this description are intended to cover both directly connected or connected through one or more intermediate means, unless otherwise stated.

The term “associated with”, used herein when referring to two elements refers to a broad relationship between the two elements. The relationship includes, but is not limited to a physical, a chemical or a biological relationship. For example, when element A is associated with element B, elements A and B may be directly or indirectly attached to each other or element A may contain element B or vice versa.

The term “adjacent” used herein when referring to two elements refers to one element being in close proximity to another element and may be but is not limited to the elements contacting each other or may further include the elements being separated by one or more further elements disposed therebetween.

The term “and/or”, e.g., “X and/or Y” is understood to mean either “X and Y” or “X or Y” and should be taken to provide explicit support for both meanings or for either meaning.

Further, in the description herein, the word “substantially” whenever used is understood to include, but not restricted to, “entirely” or “completely” and the like. In addition, terms such as “comprising”, “comprise”, and the like whenever used, are intended to be non-restricting descriptive language in that they broadly include elements/components recited after such terms, in addition to other components not explicitly recited. For example, when “comprising” is used, reference to a “one” feature is also intended to be a reference to “at least one” of that feature. Terms such as “consisting”, “consist”, and the like, may in the appropriate context, be considered as a subset of terms such as “comprising”, “comprise”, and the like. Therefore, in embodiments disclosed herein using the terms such as “comprising”, “comprise”, and the like, it will be appreciated that these embodiments provide teaching for corresponding embodiments using terms such as “consisting”, “consist”, and the like. Further, terms such as “about”, “approximately” and the like whenever used, typically means a reasonable variation, for example a variation of +/−5% of the disclosed value, or a variance of 4% of the disclosed value, or a variance of 3% of the disclosed value, a variance of 2% of the disclosed value or a variance of 1% of the disclosed value.

Furthermore, in the description herein, certain values may be disclosed in a range. The values showing the end points of a range are intended to illustrate a preferred range. Whenever a range has been described, it is intended that the range covers and teaches all possible sub-ranges as well as individual numerical values within that range. That is, the end points of a range should not be interpreted as inflexible limitations. For example, a description of a range of 1% to 5% is intended to have specifically disclosed sub-ranges 1% to 2%, 1% to 3%, 1% to 4%, 2% to 3% etc., as well as individually, values within that range such as 1%, 2%, 3%, 4% and 5%. The intention of the above specific disclosure is applicable to any depth/breadth of a range.

Additionally, when describing some embodiments, the disclosure may have disclosed a method and/or process as a particular sequence of steps. However, unless otherwise required, it will be appreciated that the method or process should not be limited to the particular sequence of steps disclosed. Other sequences of steps may be possible. The particular order of the steps disclosed herein should not be construed as undue limitations. Unless otherwise required, a method and/or process disclosed herein should not be limited to the steps being carried out in the order written. The sequence of steps may be varied and still remain within the scope of the disclosure.

Furthermore, it will be appreciated that while the present disclosure provides embodiments having one or more of the features/characteristics discussed herein, one or more of these features/characteristics may also be disclaimed in other alternative embodiments and the present disclosure provides support for such disclaimers and these associated alternative embodiments.

### DESCRIPTION OF EMBODIMENTS

The present invention provides a method of degrading unwanted nucleic acids in a sample along with rapid creation of DNA and RNA libraries in a cost-effective manner so that detecting non-host species in a host sample can be accurately and rapidly performed. Such techniques enable widespread use of next generation metagenomic sequencing, particularly for pathogen detection in humans.

The first part of the Description focuses on degrading unwanted nucleic acids in a sample by contacting the sample with magnetic particles coupled to enzymes to degrade unwanted nucleic acids present in the sample. The magnetic particles coupled to enzymes are capable of degrading both DNA and RNA. Application of a magnetic field removes the magnetic particles coupled to enzymes from the sample.

The second part of the Description focuses on one-pot synthesis of a DNA/RNA library from the degraded sample. The two techniques, used together in, for example, metagenomics sequencing, allowing for detection of pathogens in an untargeted manner. That is, pathogens are detected without an a priori guess of what pathogens might be present in the samples. Together, the techniques enable rapid and cost-effective performance of metagenomics sequencing.

### Degrading Unwanted Nucleic Acids in a Sample

Exemplary, non-limiting embodiments of a method of degrading unwanted nucleic acids in a sample, a method of detecting non-host species in a host sample and associated compositions and methods are disclosed hereinafter.

There is provided a method of degrading unwanted nucleic acids in a sample. In various embodiments, the method comprises contacting/incubating the sample with magnetic particles coupled to enzymes to degrade unwanted nucleic acids present in the sample, wherein the magnetic particles coupled to enzymes are capable of degrading DNA, RNA or both DNA and RNA. Advantageously, in various embodiments, the unwanted nucleic acids comprise free/unprotected nucleic acids that may be selectively degraded by the enzymes to deplete free/unprotected nucleic acids in the sample.

In various embodiments, the degradation or digestion of the unwanted nucleic acids is carried out in a manner or to an extent where signals from these unwanted nucleic acids are substantially removed or reduced when the sample is subsequently processed for nucleic acid detection. For example, after the unwanted nucleic acids are substantially degraded or digested, they substantially do not negatively interfere with the detection of the presence or identity of other nucleic acids of interest e.g., a good signal-to-background ratio of the nucleic acids of interest may be obtained e.g., during high throughput sequencing.

In various embodiments, the method is capable of removing at least about 50%, at least about 55%, at least about 60%, at least about 65%, at least about 70%, at least about 75%, at least about 80%, at least about 85%, at least about 90%, at least about 95%, at least about 99% or at least about 100% of the unwanted nucleic acids. In various embodiments, the method is capable of reducing the signal from the unwanted nucleic acids by at least about 50%, at least about 55%, at least about 60%, at least about 65%, at least about 70%, at least about 75%, at least about 80%, at least about 85%, at least about 90%, at least about 95%, at least about 99% or at least about 100%.

In various embodiments, the method is capable of enriching the nucleic acids of interest by at least about 10%, at least about 20%, at least about 30%, at least about 40%, at least about 50%, at least about 60%, at least about 70%, at least about 80%, at least about 90%, at least about 100%, at least about 1-fold, at least about 2-fold, at least about 3-folds, at least about 4-folds, at least about 5-folds, at least about 6-folds, at least about 7-folds, at least about 8-folds, at least about 9-folds or least about 10 folds. In various embodiments, the method is capable of increasing the signal of the nucleic acids of interest by at least about 10%, at least about 20%, at least about 30%, at least about 40%, at least about 50%, at least about 60%, at least about 70%, at least about 80%, at least about 90%, at least about 100%, at least about 1-fold, at least about 2-fold, at least about 3-folds, at least about 4-folds, at least about 5-folds, at least about 6-folds, at least about 7-folds, at least about 8-folds, at least about 9-folds or least about 10 folds.

In various embodiments, the unwanted nucleic acids comprise DNA, RNA or a combination thereof. The DNA and RNA may be single-stranded or double-stranded. These DNA and RNA may be the original full-length DNA or RNA that resides in the organism/cell that it is derived from, or they may be shortened portions/fragments of the original length DNA or RNA.

In various embodiments, the unwanted nucleic acids are derived/originated/released from the same species. In various embodiments, the unwanted nucleic acids consist of nucleic acids derived/originated/released from one species. For example, where the nucleic acids of interest comprise those derived/originated/released from non-host species, the unwanted nucleic acids may comprise those derived/originated/released from a host species. Examples of host and non-host species include but are not limited to animals, plants, microorganisms, protozoa, fungi, bacteria, virus and parasites. Bacteria include gram-negative bacteria and gram-positive bacteria. Examples of gram-negative bacteria include, but are not limited to, Escherichia coli, Pseudomonas, Salmonella, Moraxella, Helicobacter, Stenotrophomonas, Bdellovibrio, Legionella and Neisseria. Examples of gram-positive bacteria include, but are not limited to Streptococci, Staphylococci, Enterococci, Bacillus, Corynebacterium, Listeria, Lactobacilli, Erysipelothrix and Clostridium. Virus include DNA virus and RNA virus. Examples of DNA virus include, but are not limited to, those from the families Adenoviridae (e.g. Canine hepatitis virus, common cold), Herpesviridae (e.g. Herpes simplex virus, varicella-zoster virus, cytomegalovirus, Epstein-Barr virus) and Poxviridae (e.g. Smallpox virus, cowpox, myxoma virus, monkeypox, vaccinia virus). Examples of RNA virus include, but are not limited to, those from the families Retroviridae (e.g., human immunodeficiency virus) Flaviviridae (e.g., Dengue virus, Hepatitis C virus), Orthomyxoviridae (influenza), and Coronaviridae (e.g., human coronavirus, SARS coronavirus, SARS-CoV-2). Fungi include those from the classes Chytridiomycota (chytrids), Zygomycota (bread molds), Ascomycota (yeasts and sac fungi) e.g., Saccharomyces, and Basidiomycota (club fungi) e.g., Cryptococcus. In various embodiments, the unwanted nucleic acids comprise a nucleic acid derived/originated/released from a host cell, a eukaryotic cell, an animal cell, a mammalian cell and/or a human cell. In various embodiments, the unwanted nucleic acids comprise host nucleic acids. In various embodiments, the unwanted nucleic acids comprise human nucleic acids. In various embodiments, the unwanted nucleic acids comprise mitochondrial and/or ribosomal RNAs. Non-limiting examples of unwanted nucleic acids include those of actin beta (ACTB), glyceraldehyde 3-phosphate dehydrogenase (GADPH), mitochondrially encoded 12S RRNA (MT-RNR1), mitochondrially encoded 16S RRNA (MT-RNR2), ribosomal protein S18 gene (RPS18) and combinations thereof. In some embodiments, the unwanted nucleic acids do not comprise a nucleic acid derived/originated/released from a non-eukaryotic cell, a prokaryotic cell, a non-animal cell, a non-mammalian cell, a non-human cell and/or a virus. In various embodiments, the unwanted nucleic acids do not include or are substantially devoid of non-host cell nucleic acids (for e.g., bacteria, fungi, protozoa, virus, parasite nucleic acids).

The unwanted nucleic acids may be protected (e.g., membrane-bound nucleic acids such as those encapsulated by or contained in vesicles, exosomes and the like) or unprotected (e.g., nucleic acids in free form, cell-free nucleic acids, extracellular nucleic acid and the like). The unwanted nucleic acids may be found in or circulating in the biological fluids (e.g., plasma, cerebrospinal fluid, nasal excretion, urine, mucus etc.) of a host subject. These unwanted nucleic acids may be released into biological fluids or the sample due to unintended lysis of cells, or due to naturally occurring processes such as cell death, apoptosis or production of vesicles, exosomes and the like for cell-to-cell communication. The unwanted nucleic acids may be isolated or may be present as part of a biological complex.

A biological complex may include any product, e.g., extracellular product, that are produced by or derived from a cell e.g., a eukaryotic cell and/or an intact cell. Thus, in various embodiments, a biological complex excludes a cell e.g., a eukaryotic cell and/or an intact cell and a virus. A biological complex also includes cellular component(s) that is freed from its originating cell, for example, due to apoptosis/death/lysis/unintended lysis of the cell. Examples of biological complexes include, but are not limited to, membrane-bound particles/molecules (e.g., extracellular membrane-bound particles/molecules), lipoproteins, extracellular vesicles, microvesicles, exosomes, organelles (e.g., mitochondria), apoptotic bodies or the like. In various embodiments, a membrane-bound particle/molecule comprises a single membrane-bound particle/molecule. In various embodiments, a membrane-bound particle/molecule comprises a lipid bilayer-enclosed particle/molecule.

In various embodiments, the enzymes comprise nucleases such as phosphodiesterase, DNAse, RNAse, or combinations thereof. In various embodiments, the nucleases are capable of degrading DNA, RNA or both DNA and RNA. In some embodiments, the nuclease is one that can serve as both a DNAse and a RNAse simultaneously i.e., one that is capable of digesting/degrading DNA and RNA. The nuclease may be an endonuclease, an exonuclease or may have both endonuclease and exonuclease activities. The nuclease may be a recombinant nuclease, a synthetic nuclease or a wild-type nuclease or naturally occurring nuclease such as one that is purified from living organisms e.g., from animals. Examples of nucleases include, but are not limited to, RNAse A, RNAse T1, RNase I, baseline-zero DNase, benzonase, DNase I, DNase II, endonuclease III, endonuclease IV, endonuclease V, endonuclease VIII, exonuclease I, exonuclease III, exonuclease V, exonuclease VII, exonuclease VIII, exonuclease T, lambda exonuclease, micrococcal nuclease, mung bean nuclease, nuclease BAL-31, nuclease P1, nuclease S1, T4 endonuclease V, T5 exonuclease, T7 endonuclease I, T7 exonuclease. In various embodiments, the nucleases are selected from the group consisting of metazoan DNAase I, RNAse A, RNAse T1, Benzonase and combinations thereof.

In various embodiments, the nuclease is capable of degrading single-stranded nucleic acids (e.g., a single-strand specific nuclease), double-stranded nucleic acids (e.g., a double-strand specific nuclease) or both single-stranded nucleic acids and double-stranded nucleic acids.

In various embodiments, the nucleases are coupled to magnetic particles. A variety of different techniques may be used to couple or conjugate the nucleases to magnetic particles. For example, magnetic particles may be treated to provide a surface coating of a polymer carrying functional group(s) and the nucleases may be conjugated to functionalised magnetic particles via covalent linkages such as click chemistry, epoxy-amine adducts, amide bond, ester bond, disulphide bond or the like. Any suitable reactive group present on nuclease and/or surface of the magnetic particles e.g., functionalized magnetic particles can be used to covalently bind the enzyme to the surface of the magnetic particles. The functional groups may include but are not limited to phosphate, phosphonate, sulfate, sulfonate, azide, alkyne, alkene, anhydride, aldehyde, amine, hydroxyl, carboxyl, tetrazine, thiol, and epoxy groups. It will be appreciated that other suitable methods to conjugate the nucleases to the magnetic particles may also be employed provided such methods can sufficiently secure the nucleases to the magnetic particles without removing the effectiveness of the nucleases to degrade nucleic acids and without removing the effectiveness of the magnetic particles to manipulated by an application of a magnetic field.

In some embodiments, a plurality of nucleases may be coupled to a single magnetic particle. The plurality of nucleases coupled to the magnetic particle may be the same type of nucleases or in the same category of nucleases (e.g., DNAse, RNAse or phosphodiesterase, e.g., single-strand specific nuclease or double-strand specific nuclease). Alternatively, the plurality of nucleases coupled to the magnetic particle may be different or from a different category of nucleases. For example, a DNAse and a RNAse may be both coupled to a single magnetic particle. This is advantageous if both DNAse and RNAse capabilities are required at the same time. For example, a single-strand specific nuclease and a double-strand specific nuclease may be both coupled to a single magnetic particle. This is advantageous if degradation of both single-stranded and double-stranded DNA/RNA are required at the same time. In some embodiments, a single nuclease or a single type of nuclease may be coupled to a single magnetic particle. This may be possible even if both DNAse and RNAse capabilities, or removal of both single-stranded and double-stranded DNA/RNA are required at the same time as the single nuclease or a single type of nuclease may be one that can simultaneously serve as a DNAse and RNAse (e.g., Benzonase and phosphodiesterase) or one that can simultaneously degrade both single-stranded and double-stranded DNA/RNA (e.g., DNAse I). Furthermore, as various embodiments utilize a plurality of magnetic particles, each magnetic particle may be coupled to nuclease(s) having different enzymatic activity from the other magnetic particles, and thus collectively being capable of providing both DNAse and RNAse activities. In various embodiments, the magnetic particles coupled to nucleases comprise a magnetic particle coupled to both a DNAse and a RNAse, a magnetic particle coupled to a nuclease capable of degrading both DNA and RNA, a mixture comprising a magnetic particle coupled to a DNAse and a magnetic particle coupled to a RNAse, or combinations thereof. Depending on the nature and/or identity of the unwanted nucleic acids (e.g., whether DNA and/or RNA, single-stranded and/or double stranded), one or more different nucleases can be suitably selected and coupled to the magnetic particles to suitably remove the unwanted nucleic acids.

In various embodiments, the method comprises applying a magnetic field to remove the magnetic particles coupled to nucleases from the sample. Advantageously, embodiments of the method are capable of efficiently removing traces of the nucleases activity by effectively removing the magnetic particles coupled to nucleases using a non-contact means such as a magnetic field. This may prevent carryover of the nucleases to a next reaction or step. Thus, various embodiments of the methods disclosed herein provide an effective way to degrade/digest unwanted nucleic acids in a sample with nucleases and subsequently removing nuclease activity by creatively employing magnetic particles coupled to nucleases and a magnetic field.

The magnetic field may be one that exerts a magnetic force on the magnetic particles to concentrate, congregate, aggregate or pool them together. The magnetic force may be attractive force or a repulsive force. In various embodiments, the magnetic force is an attractive force to attract and concentrate the magnetic particles together. In various embodiments, the sample is then removed from magnetic particles by removing the solution/buffer solution/supernatant (e.g., by pouring or pipetting) from the magnetic particles that remain attracted and concentrated by the magnetic field at a specific part of a receptacle/container (e.g., a test tube) that holds the sample. Alternatively, the magnetic particles may be directly removed from the sample by moving the magnetic particles out of the sample using the attractive forces imparted by the magnetic field. In various embodiments, as the nucleases are coupled to the magnetic particles, removal of the magnetic particles from the sample or vice versa would result in the sample/solution/buffer solution/supernatant being substantially free from the added nucleases.

In various embodiments, the magnetic field is applied through the use of a magnet. The magnet may be a permanent magnet or an electromagnet. For example, the magnet may be a permanent magnet, or the magnet may be an electromagnet comprising coils embedded within the base member and connected to an external power supply. A combination of different magnet types may also be employed. For the case where the magnet is a permanent magnet, the system may be capable of performing the assay without an external power source. In various embodiments, the magnet employed is in the form of a magnetic rack.

The unwanted nucleic acids may be as part of a biological complex. For example, the unwanted nucleic acids may be contained in membrane-bound particles/molecules, optionally a single membrane-bound particles/molecules (e.g., extracellular membrane-bound particles/molecules), lipoproteins, extracellular vesicles, microvesicles, exosomes, organelles (e.g., mitochondria), apoptotic bodies etc. For example, the unwanted nucleic acids may be encased/encapsulated in a lipid bilayer, optionally a single lipid bilayer. In various embodiments therefore, the method further comprises applying a lysing agent to the sample for selectively lysing biological complexes that contain said unwanted nucleic acids in the sample to free said unwanted nucleic acid for degradation by the nucleases. The step allows unwanted nucleic acids that reside in biological complexes to be freed into the sample for degradation by the nucleases. Advantageously, this may allow for a cleaner and more comprehensive removal of the unwanted nucleic acids that may also add to the background noise during detection of other nucleic acid of interest.

In some embodiments, the application of a lysing agent may not be required, particularly if the method is being deployed in separate use cases in which selective lysis is not required. For example, selective enrichment of specific species of cells or biological complexes such as exosomes from complex biological fluids such as blood may result in unintended lysis of cells, thus releasing nucleic acids into the solution. Thus, the nucleases-coupled magnetic particles/beads may be applied to the bulk mix to degrade ‘ambient’ nucleic acids without the need to wash the cells and the particles/beads can then be removed easily with a magnet without a wash step. Advantageously, this allows for an effective removal of the ‘ambient’ nucleic acids which may otherwise be captured with the signal nucleic acids in subsequent purification steps and adversely affect the precise measurement of specific nucleic acid signals.

In various embodiments, the lysing agent is substantially incapable of lysing cells/viruses protected by a cell wall (e.g., bacteria and fungi), a double membrane (e.g., parasites) and/or a protein coat (e.g., viruses and protozoa protected by a pellicle) to free the nucleic acids contained therein for degradation by the magnetic particles coupled to nucleases. In various embodiments, the lysing agent is substantially incapable of lysing viruses and/or intact cells e.g., intact cells selected from bacteria, protozoa, fungi, parasites and combinations thereof to free the nucleic acids contained therein for degradation by the magnetic particles coupled to nucleases. In various embodiments, the intact cells and/or viruses contain nucleic acids of interest. Therefore, in various embodiments, the intact cells and/or viruses are not actively lysed by the lysing agent so as to prevent leakage of the nucleic acids of interest into the sample and their subsequent undesired degradation by the nucleases even before these nucleic acids of interest are detected and identified. In some embodiments, the intact cells may not be containing nucleic acid of interest but nevertheless will not substantially contribute to the background noise during detection and identification of nucleic acids of interest; thus, it may not be necessary to release and degrade the nucleic acid contained in these intact cells. Accordingly, in various embodiments, the lysing agent is a selective lysing agent that only selectively lyses free biological complexes that contain the unwanted nucleic acids and that may contribute to the background noise during detection and identification of other nucleic acids of interest. Advantageously, when the nucleic acids of interest are contained in non-host species/organisms such as bacteria, fungi, protozoa, parasites and viruses, the method may be capable of leaving cell-wall protected bacterial/fungal nucleic acids, double membrane-protected parasite nucleic acids and protein-protected viruses/protozoa intact.

In various embodiments, the lysing agent comprise a mild detergent. The mild detergent may be one that is capable of lysing free biological complexes but not intact cells or viruses.

In various embodiments, the sample may be substantially free of or devoid of intact cells containing the unwanted nucleic acids prior to the step of contacting/incubating the sample with magnetic particles coupled to nucleases. For example, if the nucleic acids of host cells are not of interest and may contribute to the background noise when detecting or identifying the nucleic acids of interest (e.g., from non-host cells), the sample may be substantially free of or devoid of intact host cells prior to the step of contacting/incubating the sample with magnetic particles coupled to nucleases. Accordingly, in various embodiments, the method further comprises a step of removing intact cells containing unwanted nucleic acids prior to the contacting/incubating step. In some embodiments, the step of removing intact cells containing unwanted nucleic acids may be carried out after the contacting/incubating step. Suitable methods of removing intact cells containing unwanted nucleic acids from the sample include centrifugation (e.g., low speed centrifugation), filtration, decanting or the like and combinations thereof.

In various embodiments, the sample comprises a biological sample. In various embodiments, the biological sample comprises a fluid biological sample or a liquid biological sample. The fluid biological sample or liquid biological sample may be blood, serum, plasma, sputum, lavage fluid, cerebrospinal fluid, urine, semen, sweat, tears, saliva, and the like. In some embodiments, the fluid biological sample or liquid biological sample comprises whole blood, blood serum, blood plasma or processed fractions thereof. In some embodiments, the fluid biological sample comprises blood serum or blood plasma. In various embodiments, the sample comprises a sample derived from a host subject and said unwanted nucleic acids comprise host nucleic acids but are substantially devoid of or do not include non-host nucleic acids. In various embodiments, the sample comprises non-host species, cellular organisms or viruses. Advantageously, the method is capable of being employed to detect non-host species residing within or on a host.

Accordingly, there is also provided a method of detecting non-host species in a host sample, the method comprising contacting/incubating the host sample with magnetic particles coupled to nucleases to degrade cell-free host nucleic acids present in the host sample, wherein the magnetic particles coupled to nucleases are capable of degrading DNA, RNA or both DNA and RNA; applying a magnetic field to remove the magnetic particles coupled to nucleases from the host sample; and detecting the presence of non-host nucleic acids in the sample. The contacting/incubating step may be conducted under similar conditions as previously described (e.g., temperature and duration). The method may further comprise removing host cells from the host sample prior to contacting/incubating the host sample with magnetic particles coupled to nucleases.

In various embodiments, the method of detecting non-host species in a host sample further comprises applying a first lysing agent to the host sample for selectively lysing cell-free host biological complexes prior to contacting/incubating the host sample with magnetic particles coupled to nucleases. The first lysing agent may be substantially incapable of lysing non-host species to free the nucleic acids contained therein for degradation by the magnetic particles coupled to nucleases. The first lysing agent may comprise a mild detergent that have similar properties (e.g., identity, properties and concentration) as those previously described.

In various embodiments, the method of detecting non-host species in a host sample further comprises applying a second lysing agent to the host sample for lysing non-host species to free the nucleic acids contained therein. In various embodiments, the second lysing agent is different from the first lysing agent. The second lysing agent may utilize a similar or a different mechanism of action from the first lysing agent to lyse the non-host species. For example, the lysing action of the second lysing agent may be non-chemical in nature. Accordingly, in various embodiments, the second lysing agent utilizes a physical or mechanical force for lysing intact cells. For example, the second lysing agent may comprise lysing particles or beads that are capable of imparting a mechanical force to lyse the non-host species and free the nucleic acids contained therein. In some examples, the lysing action of the second lysing agent may be chemical in nature. For example, the second lysing agent may comprise protease such as proteinase K or the like to lyse the non-host species and free the nucleic acids contained therein. It will be appreciated that, depending on the identity and nature of the non-host species that are to be lysed, other suitable lysing agent may also be employed to free the nucleic acids contained therein.

In various embodiments, the lysing particles or beads comprises inorganic particles such as silica particles or the like; inorganic metal oxide particles, such as alumina, titania, zirconia, ceria or the like; or combinations thereof. The lysing particles or beads may have an average particle size falling in the range of from about 100 μm to about 1000 μm, from about 150 μm to about 950 μm, from about 200 μm to about 900 μm; from about 250 μm to about 850 μm; from about 300 μm to about 800 μm; from about 350 μm to about 750 μm; from about 400 μm to about 700 μm; from about 450 μm to about 650 μm; from about 500 μm to about 600 μm; or from about 500 μm to about 550 μm. In various embodiments, the lysing particles have an average particle size of about 500 μm.

In various embodiments, the method of detecting non-host species in a host sample further comprises applying an amplification process to the host sample for amplifying the nucleic acids freed from the non-host species. Amplification reactions known in the art may be employed. The amplification reactions may include but are not limited to polymerase chain reaction (PCR), ligase chain reaction (LCR), loop mediated isothermal amplification (LAMP), nucleic acid sequence-based amplification (NASBA), self-sustained sequence replication (3SR), rolling circle amplification (RCA) or any other process whereby one or more copies of a particular nucleic acid sequence may be generated from a nucleic acid template sequence. Reverse transcription to generate a complementary strand of DNA (cDNA) from RNA may be carried out prior to amplification.

In various embodiments, the method of detecting non-host species in a host sample further comprises detecting the presence and/or identity of the non-host nucleic acids which may already be amplified. Detection of a presence and/or identity of the non-host nucleic acids may be carried out according to any one of the many methods available to the man skilled in the art. In various embodiments, the detection may be carried out by performing assays that may include but is not limited to DNA sequencing methods, next-generation sequencing (NGS) methods, whole genome sequencing (WGS) methods, whole exome sequencing (WES) methods, panel sequencing methods, paired-end sequencing methods, DNA microarray methods or the like. Reverse transcription to generate a complementary strand of DNA (cDNA) from RNA may be carried out during the detecting step.

The method may be also be useful for detecting infection in a subject. In various embodiments therefore, there is provided a method of detecting infection in a subject, the method comprising: contacting a sample from the subject with magnetic particles coupled to nucleases to degrade cell-free host nucleic acids present in the sample optionally released by a lysing agent such as a mild detergent, wherein the magnetic particles coupled to nucleases are capable of degrading both DNA and RNA; applying a magnetic field to remove the magnetic particles coupled to nucleases from the sample; and detecting the presence of non-host nucleic acids in the sample. In various embodiments, the detection of the presence of non-host nucleic acids in the sample is indicative that the subject has an infection. In various embodiments, the infection is selected from the group consisting of bacterial infection, fungi infection, protozoan infection, parasitic infection, viral infection and combinations thereof. In various embodiments, the method further comprises administering to the subject anti-infectives such as antibacterial, antivirals, antifungals and antiparasitic medications/drugs when the subject is indicative to have an infection. In various embodiments, the method comprises a diagnostic method.

The method may also be useful for determining/evaluating/monitoring the progression of an infection in a subject. In various embodiments, there is provided a method of determining/evaluating/monitoring the progression of an infection in a subject, the method comprising: a) contacting a first sample from the subject with magnetic particles coupled to nucleases to degrade cell-free host nucleic acids present in the first sample, wherein the magnetic particles coupled to nucleases are capable of degrading both DNA and RNA; b) applying a magnetic field to remove the magnetic particles coupled to nucleases from the first sample; c) detecting the presence and/or amount of non-host nucleic acids in the first sample; d) repeating steps a) to c) for a second sample from the subject, and e) comparing the amount of the non-host nucleic acids in the first sample and the second sample, wherein the first sample is obtained from the subject at an earlier timepoint (e.g. days or weeks before) than the second sample. In various embodiments, where the second sample is found to contain a less amount of the non-host species as compared to the first sample, this is indicative of an improvement of the infection in the subject. In various embodiments, where the second sample is found to contain a substantially the same or a greater amount of the non-host species as compared to the second sample, this is indicative of a non-improvement or a worsening of the infection in the subject. In various embodiments, the method comprises a prognosis method. For example, where the second sample is found to contain a less amount of the non-host species as compared to the first sample, this may be indicative of a good prognosis in the subject.

Advantageously, embodiments of the method are capable of reducing/eliminating background signal and/or increasing/enhancing the signal of the nucleic acids of interest. In various embodiments therefore, embodiments of the method show improved/high sensitivity for detecting an infection/presence of non-host species in a subject.

The degradation of cell-free nucleic acids such as RNA and/or DNA may be useful in single-cell sequencing preparation. For example, in a single cell transcriptomic or genomic assay, any cell-free nucleic acid that gets incorporated into an emulsion is also tagged with a unique cell-specific barcode, which is undesirable. Thus, a first or early removal of cell-free nucleic acid(s) in a single-cell analysis/sequencing method may advantageously eliminate/reduce contamination by any cell-free nucleic acid(s), thereby improving the accuracy of the single-cell analysis/sequencing result. In various embodiments therefore, there is provided a method of single-cell analysis or sequencing, the method comprising: contacting a sample comprising a single cell with magnetic particles coupled to nucleases to degrade cell-free nucleic acids present in the sample, wherein the magnetic particles coupled to nucleases are capable of degrading both DNA and RNA; applying a magnetic field to remove the magnetic particles coupled to nucleases from the sample; and sequencing nucleic acids from the single cell. In various embodiments, the method further comprises a step of amplifying the nucleic acids released from the single cell prior to the sequencing step. In various embodiments therefore, sequencing nucleic acids from the single cell comprises sequencing the amplicons of the nucleic acids from the single cell. In various embodiments, amplifying the nucleic acids released from the single cell comprises amplifying the nucleic acids with a primer comprising a barcode sequence to obtain amplified nucleic acids comprising the barcode sequence. Where a plurality of single cells is sequenced, the barcode sequence may allow for the identification of the single cell which an amplified nucleic acid originate from. For example, primers each comprising a unique cell-specific barcode sequence may be used to amplify nucleic acids originating from different single cells so that the origin of an amplified nucleic acid can be traced based on the unique cell-specific barcode sequence tagged thereto.

In various embodiments, the method comprises an in vivo, ex vivo or an in vitro method.

There is also provided a magnetic particle for use in embodiments of the methods disclosed herein. In various embodiments, the magnetic particle comprises one or more nucleases coupled thereto. The magnetic particle and nuclease(s) coupled thereto may comprise one or more of the properties previously described. In some embodiments, the magnetic particle is configured to provide DNAse and RNAse activities.

In various embodiments, there is provided a composition comprising a plurality of magnetic particles coupled to nucleases, said plurality of magnetic particles capable of degrading RNA, DNA or both RNA and DNA. In various embodiments, the plurality of magnetic particles comprise a magnetic particle coupled to both a DNAse and a RNAse, a magnetic particle coupled to a nuclease capable of degrading both DNA and RNA, a mixture comprising a magnetic particle coupled to a DNAse and a magnetic particle coupled to a RNAse, or combinations thereof. The magnetic particles and/or nucleases may comprise one or more of the properties previously described. In various embodiments, the composition comprises a plurality of the magnetic particles coupled to nucleases in a buffer solution. In various embodiments, the buffer solution has a substantially neutral or slightly alkaline pH. In various embodiments, the buffer solution has a pH of from about 5 to about 9, from about 6 to about 8 or from about 7 to about 8. In one embodiment, the pH of the buffer solution is about 7.5. The buffer solution may comprise salts such as Tris-HCl, calcium chloride, sodium chloride, magnesium chloride and other suitable salts.

In various embodiments, there is provided a kit comprising the composition, a first lysing agent and/or a second lysing agent. The first lysing agent and second lysing agent may comprise one or more of the properties previously described.

In various embodiments, there is provided a product or a method as described herein.

### One-Pot Synthesis of DNA and RNA Libraries

The following description relates to certain preferred embodiments for performing one-pot synthesis of DNA and RNA libraries. However, it is understood that other one-pot synthesis techniques for creating DNA and RNA libraries may be used in the pathogen detection techniques of the present invention.

In the following description, a simultaneous amplification method for generating a library from a mix of different nucleic acids without bias is set forth as preferred examples. It will be apparent to those skilled in the art that modifications, including additions and/or substitutions may be made without departing from the scope and spirit of the invention. Specific details may be omitted so as not to obscure the invention; however, the disclosure is written to enable one skilled in the art to practice the teachings herein without undue experimentation.

An unbiased and simultaneous amplification method for preparing a library from a sample of more than one type of nucleic acid in substantially low amount comparative to non-nucleic acid molecules, where the method includes:
providing the sample of more than one type of nucleic acid including single-stranded and/or double-stranded DNA and/or RNA as a template of subsequent extensions and amplifications;
preparing a first DNA strand from said sample including annealing one or more first DNA strand generation primers to any of the DNA and/or RNA template, and extending from the annealed first DNA strand generation primer including employing a DNA polymerase that enables a one-pot synthesis of the first DNA strand from either or both of DNA and/or RNA templates to obtain the first DNA strand, wherein each of the first DNA strand generation primers includes:
a first nucleotide sequence that includes a constant adaptor sequence on the 5′ end followed by a poly-thymidine sequence and two random nucleotides, said poly-thymidine sequence including at least ten thymidine bases followed by a random nucleotide selected from a set of adenine, cytosine or guanine and further followed by a random nucleotide selected from a set of four bases of adenine, cytosine, guanine and thymidine (SEQ ID NO: 18), and a second nucleotide sequence that includes a constant adaptor sequence on the 5′ end followed by five, six or seven random nucleotides, said random nucleotide being a nucleotide base randomly selected from a set of any three bases of adenine, cytosine, guanine and thymidine, or a combination thereof;
preparing a second strand of DNA or DNA fragment including annealing a second DNA strand generation primer to the first DNA strand after dissociation from the DNA and/or RNA template and denaturing thereof, and extending from the annealed second DNA strand generation primer including employing a DNA polymerase having strand displacement activity, wherein said second DNA strand generation primer includes:
   a random nucleotide sequence including a plurality of said random nucleotides at 3′-end of the second strand of DNA, and
   a second adaptor sequence at 5′-end thereof that is physically linked to the random nucleotide sequence,
   wherein the plurality of said random nucleotides includes at least five random nucleotides wherein each of said at least five random nucleotides is selected from a set of any three bases of adenine, cytosine, guanine and thymidine;
amplifying the second strand of DNA or DNA fragment via a polymerase chain reaction including annealing a pair of amplification primers including the first adaptor sequence at 5′-end in one of the amplification primers and the second adaptor sequence at 5′-end in another one of the amplification primers to the second strand of DNA or DNA fragment to obtain a plurality of amplicons such that each of the amplicons includes at least the first and second adaptor sequences, wherein each of the first and second adaptor sequences has at least one nucleotide modified by methylation;
fragmenting the amplicons into a plurality of double-stranded DNA fragments including reacting the amplicons with a methylation-specific restriction enzyme in order to obtain the double-stranded DNA fragments absent the first and second adaptor sequences.

The random nucleotide may be selected from a set of cytosine, guanine and thymidine, a set of adenine, guanine and thymidine, a set of adenine, cytosine and thymidine, a set of adenine, cytosine and guanine, or a set of adenine, guanine, cytosine and thymidine.

In another specific embodiment, each of the random nucleotides in the first strand generation primers is jointly or independently selected from B, D, H, or V according to IUPAC nucleotide code.

In yet another specific embodiment, each of the random nucleotides in the second strand generation primers is jointly or independently selected from B, D, H, or V according to IUPAC nucleotide code.

In a more specific embodiment, the random nucleotide sequence of the first strand generation primers and/or second strand generation primers includes six to eight random nucleotides where the last random nucleotide may be different from the rest of the random nucleotides in the random nucleotide sequence according to IUPAC nucleotide code.

In another more specific embodiment, the random nucleotide sequence of the first strand generation primers and/or second strand generation primers includes six to eight random nucleotides where except the last random nucleotide, the rest of the random nucleotides in the random nucleotide sequence are jointly selected from one of the random nucleotides with random selection of three out of four nucleotide bases according to the IUPAC nucleotide code.

In another embodiment, a methylated nucleoside triphosphate is added into a reaction mixture of the first and/or second DNA strand or DNA fragment preparation(s) and/or the polymerase chain reaction.

In a preferred embodiment, wherein the methylated nucleoside triphosphate is deoxy-methyl-cytidine triphosphate in a concentration of 0.01% to 25% to result in 0.01 to 25% of cytosines in the product being methylated after the polymerase chain reaction.

In other embodiment, one cytosine in each of the first and second adaptor sequences is modified by methylation to become methyl-cytosine in the amplification primers in order to obtain a plurality of amplicons with methylated nucleotide bases.

In yet another embodiment, the present method further includes appending at least a pair of double-stranded adaptors, wherein one strand thereof comprises a 4-random nucleotide overhang complementary to a 4-nucleotide overhang on both ends of the double-strand DNA fragments after said fragmenting in order to obtain double-stranded DNA fragments under 1000 nucleotide in size containing the double-stranded adaptors appended on both ends thereof.

In a further embodiment, the double-stranded DNA fragments having been appended with the corresponding double-stranded adaptors can be further appended with a pair of sequencing adaptors on both ends thereof for an optional subsequent barcoding.

In another embodiment, the double-stranded DNA fragments having been appended with the corresponding double-stranded adaptors are amplified by annealing a pair of primers to generate more amplicons thereof before appending a pair of sequencing adaptors if subsequent barcoding is intended.

A second aspect of the present invention relates to a kit for generating a library according to the method described herein. The kit includes a substrate conjugated with two of the first strand generation primers for annealing RNA template include single-stranded RNA template and single-stranded mRNA. The kit also includes reverse transcriptase, an RNAse, and reaction mixture for initiating reverse transcription of the first DNA strand from the annealed first strand generation primers. The kit further includes the second strand generation primer, a polymerase with strand displacement activity, and reaction mixture for polymerization of the second DNA strand from the first DNA strand after separating the substrate from the liquid after the reverse transcription. The kit additionally includes amplification primers, enzyme and reaction mixture for amplification of the second DNA strand where the amplification primers correspond to the adaptor sequence in the first strand generation primers and the second strand generation primer, respectively. Amplification products after the PCR amplification of the second DNA strand can be collected from the liquid phase of the reaction product.

The followings are some of the advantages of using the present method to generate library preparation for sequencing:
(1) In the first strand generation, the first strand is generated from total nucleic acid using a melt protocol which enables binding of the random sequence to both DNA and RNA molecules. Using a specific reverse transcriptase and adaptor sequence in the first strand generation primers to generate the first DNA strand allows a one-pot synthesis of the first strand DNA template from a mixture of different nucleic acids so to avoid bias;
(2) In the second strand generation, the second strand generation primer contains a random sequence to avoid concatemer formation during the second strand generation from the first DNA strand. Since the first DNA strand contains the adaptor sequence, the complementary adaptor sequence in the second strand generation primer results in a product that has a unique identifier on either end of the second DNA strand;
(3) Inclusion of a methylated dCT into the amplification primers which contain both the adaptor sequences of the first and second strand generation primers and addition of deoxy-methylated cytosine triphosphate into the PCR reaction mixture allows the subsequent restriction enzymatic digestion specific to those amplicons at the methylated base in order to reduce the size of the fragments under 1000 nucleotide;
(4) Appending adaptor sequence with 4 nt 5′ overhangs allow later ligation of adaptors on both ends of the DNA fragments for subsequent sequencing PCR and barcoding.

### EXAMPLES

Example embodiments of the disclosure will be better understood and readily apparent to one of ordinary skill in the art from the following discussions and if applicable, in conjunction with the figures. It should be appreciated that other modifications related to structural, chemical and biological changes may be made without deviating from the scope of the invention. Example embodiments are not necessarily mutually exclusive as some may be combined with one or more embodiments to form new exemplary embodiments. The example embodiments should not be construed as limiting the scope of the disclosure.

### Example 1—Overview of a Methodology of Removing Unwanted Nucleic Acids in a Sample in Accordance with an Exemplary Embodiment Disclosed Herein

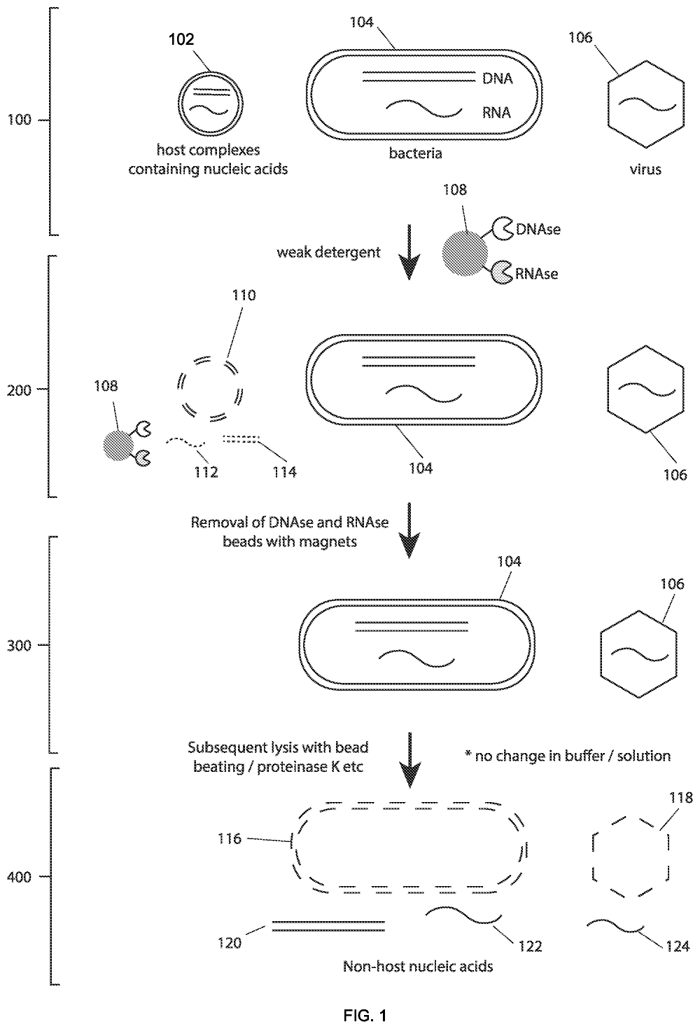

Referring to FIG. 1, there is shown a schematic flow diagram showing various process steps in accordance with an exemplary embodiment of the method disclosed herein. In this example, it is desirable to detect the presence and/or identity of non-host species (e.g., bacteria and virus) residing in a host sample. Therefore, in this example, it is desirable to remove or degrade unwanted nucleic acids, that is, extracellular host nucleic acids in the host sample prior to detection and identification of the nucleic acids of the non-host species.

In step 100, a sample obtained from a host is provided. The sample contains free host complexes 102 (e.g., lipoprotein complexes, extracellular vesicles, exosomes, organelles, apoptotic bodies or the like), bacteria 104 and virus 106. The host complexes 102, bacteria 104 and virus 106 contain nucleic acids in the form of DNA and RNA. Double-stranded DNA is represented by straight parallel lines while single-stranded RNA is represented by a wavy line.

In step 200, magnetic beads 108 conjugated to nucleases (i.e., both DNAase and RNAse in this example) are incubated with the host sample in the presence of a lysing agent which is in the form of a weak detergent. The weak detergent selectively lyses the free host complexes 102. The lysed host complexes 110 release the host RNA and DNA contained therein, which are then digested or degraded by the magnetic beads 108 conjugated to DNAase and RNAse. The degraded host DNA 114 and RNA 112 do not substantially affect the detection of the non-host nucleic acids in subsequent steps. The weak detergent is substantially incapable of degrading/digesting the bacteria 104 and the virus 106 to release the non-host nucleic acids contained therein.

In step 300, the magnetic beads 108 that are conjugated to DNAse and RNAse are removed from the host sample with magnets that provide the magnetic force.

In step 400, another lysing agent in the form of lysing beads and/or proteinase K is added to the sample. There is no change in the buffer solution that is already present in the sample in the earlier steps. This lysing agent serves to lyse non-host species such as bacteria 104 and virus 106 to result in lysed bacteria 116 and lysed virus 118. The lysed bacteria 116 and lysed virus 118 then allow the non-host nucleic acids contained therein (120, 122 and 124) to be freed and leak out into the sample for further processing and identification.

### Example 2—Exemplary Implementation of an Embodiment of the Method Disclosed Herein

To improve detection sensitivity of non-host cells nucleic acids (e.g., including those from both DNA and RNA viruses) in a host sample, both non-host DNA and RNA should be harvested to give maximum signal. Using a weak detergent for selective lysis of host complexes, combined with DNAse and RNAse degradation of host nucleic acids should result in selective depletion of host nucleic acid, but removal of DNAse and RNAse from the sample prior to non-host organism lysis with methods such as bead beating is difficult, especially as RNAse is a very tenacious enzyme. This example shows that by utilizing magnetic particles conjugated to nucleases such as DNAse and RNAse, degradation of free host nucleases in the sample is effective and the nucleases can be easily removed from the host sample.

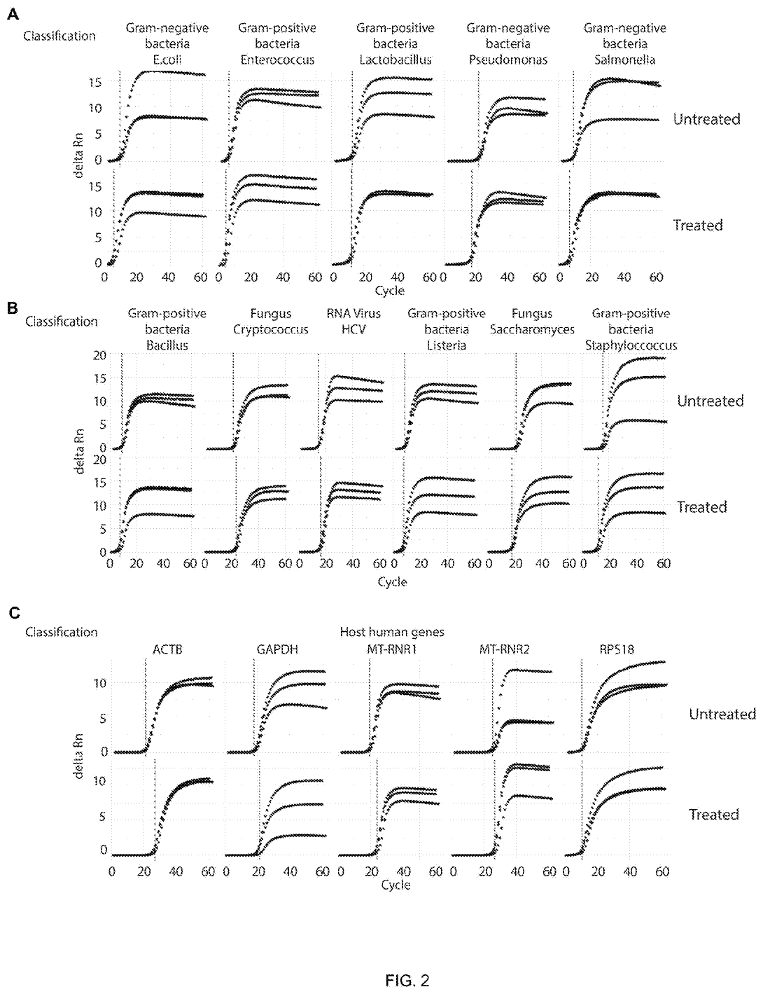

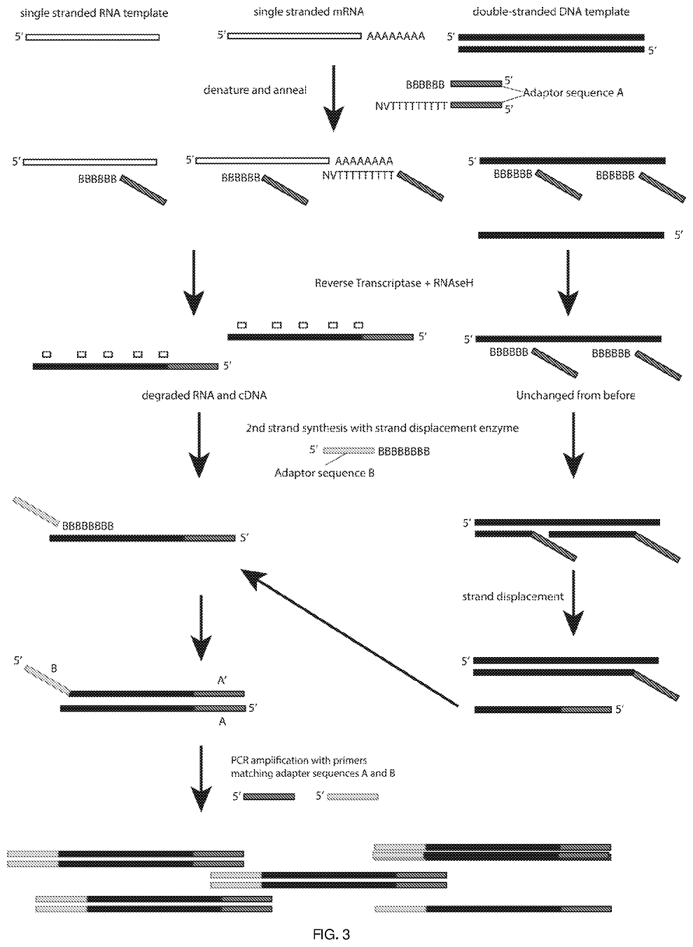

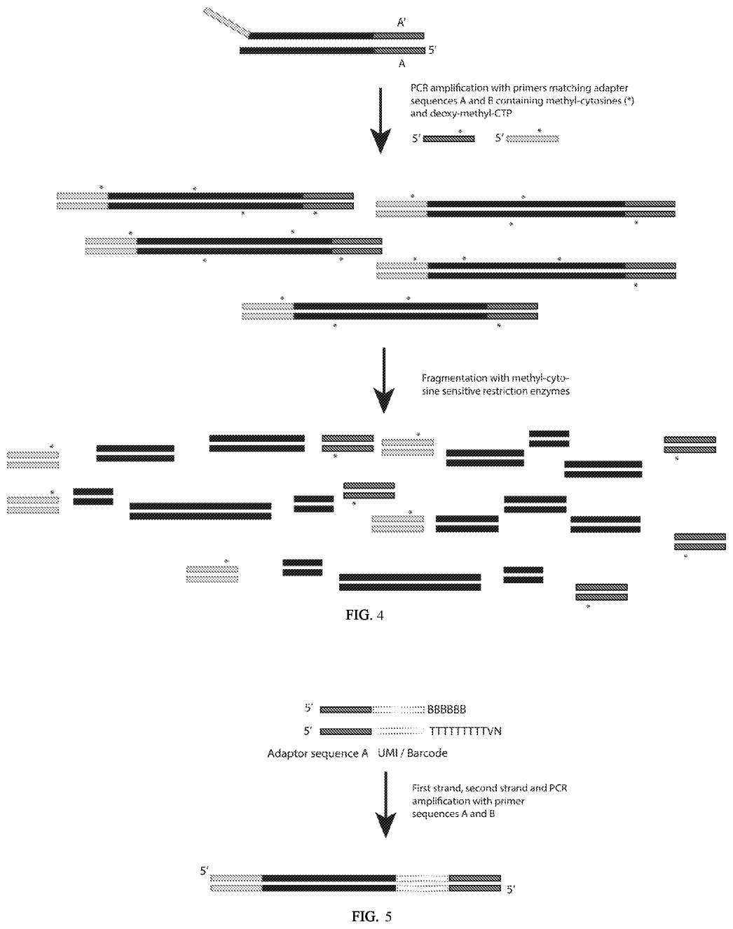

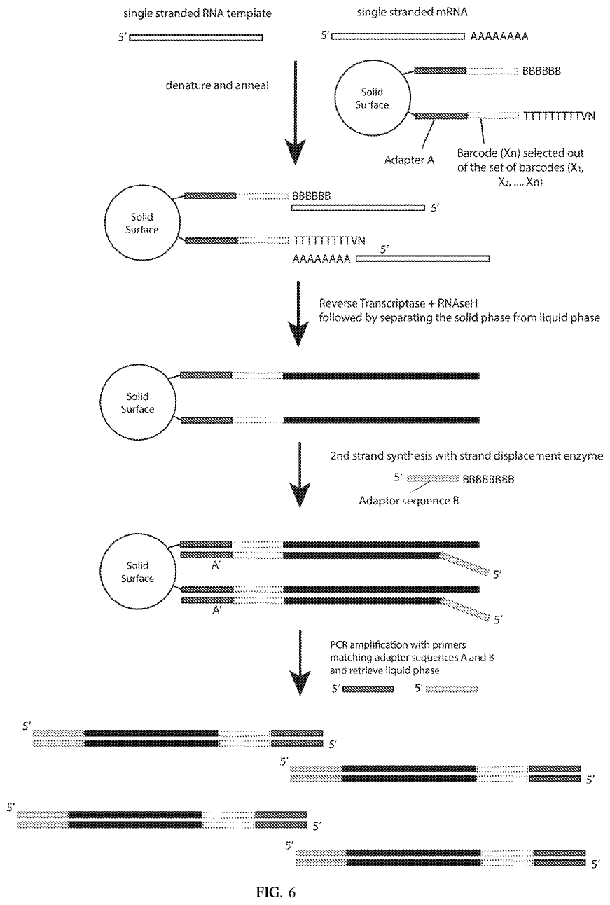

The resultant amplification plot (FIG. 2) shows the amplification curve of PCR products using primers specific for each organism or representative human genes. As PCR presumably doubles product every cycle, the cycle number (x-axis) reflects the logarithmic abundance of each product, with left-shifted amplification curves reflecting higher signal than right-shifted amplification curves. The straight vertical line drawn through each plot is the calculated Ct value corresponding to the predicted cycle number for a fixed fluorescence intensity and can be considered a reflection of the relative abundance of each species.

Comparing the Ct value for bacterial, fungal and viral components, it is clear these signals were largely the same or enhanced by the bead treatment while the host human components were largely depleted. This demonstrates that this method clearly preferentially degrades host background signal, enriching the relative contribution of non-host bacterial and fungal signals.

### Example 3—Library Preparation from HEK293 Cells

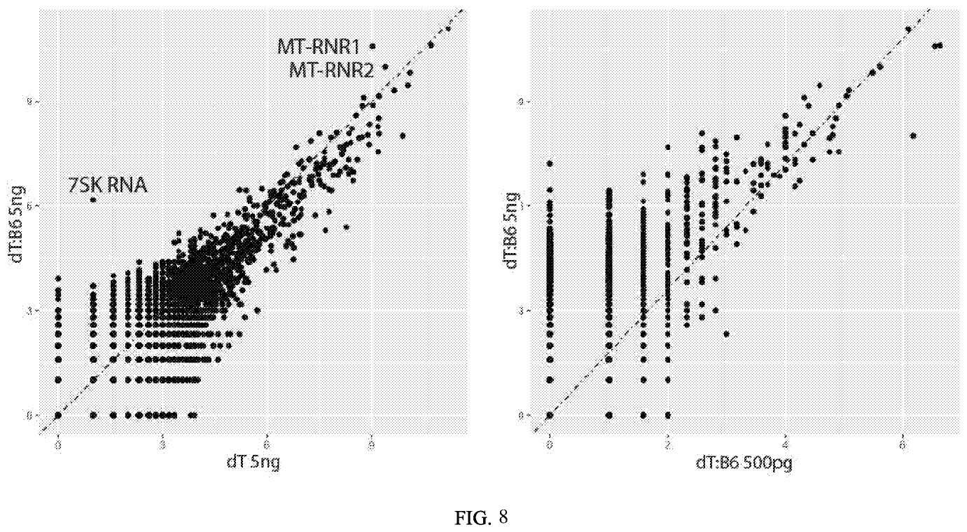

10 ng or Ing of DNAse I-treated RNA harvested from HEK293 cells was mixed with 500 nM of adaptor A-B6 (5′-CAGACTACCATGACCTGAGTCBBBBBB-3′) (SEQ ID NO: 1) and adaptor A-oligodT (5′-CAGACTACCATGACCTGAGTCTTTTTTTTTTTTTTTVN-3′) (SEQ ID NO: 2) in a 1:1 ratio or adaptor A-oligodT alone in a 10 μl reverse transcription reaction mix using PROMEGA MMLV reverse transcriptase as per manufacturer's instructions at 40° C. for 15 minutes before denaturing at 70° C. for 5 minutes. 5 μl was stored for qPCR. 1 μl of 10 μM Adaptor B-B8 (5′-GTCAGAGTCGAATGCGTACTGBBBBBBBB-3′) (SEQ ID NO: 3) was added to the other 5 of the RT reaction mix and heated to 70° C. for 2 minutes before cooling to 4° C. in the thermocycler. 1.5 μl Isothermal Buffer, 1.0 μl 25 mM MgSO₄, 2.0 μl 2 mM dNTPs, 3.5 μl water and 1 μl Bst3.0 were then added and incubated in the thermocycler at 45° C. for 20s, increasing 1° C. every 20s to 60° C. and held at 60° C. for 12 minutes. 2.25 μl 10 μM of methylated Adaptor A primer (5′-CAGACTACmCATGACCTGAGTC-3′) (SEQ ID NO: 4) and methylated Adaptor B primer (5′GTCAGAGTmCGAATGCGTACTG-3′) (SEQ ID NO: 5), 4.5 μl 2 mM dNTPs, 0.9 μl 1 μM dmCTP, 3.6 μl KOD buffer, 2.25 μl 25 mM MgSO4, 0.45 μl KOD was added and topped up with water to 45 μl. The template was then heated to 95° C. for 2 minutes, then cycled between 95° C. for 10s, 58° C. for 10s, 70° C. for 2 minutes for 10 cycles. The resultant 50 μl reaction mixture was then cleaned up with 1.0× Ampure XP beads as per manufacturer's instructions and eluted in 10 μl (FIG. 8).

### Example 4—qPCR for Normalization of Input RNA

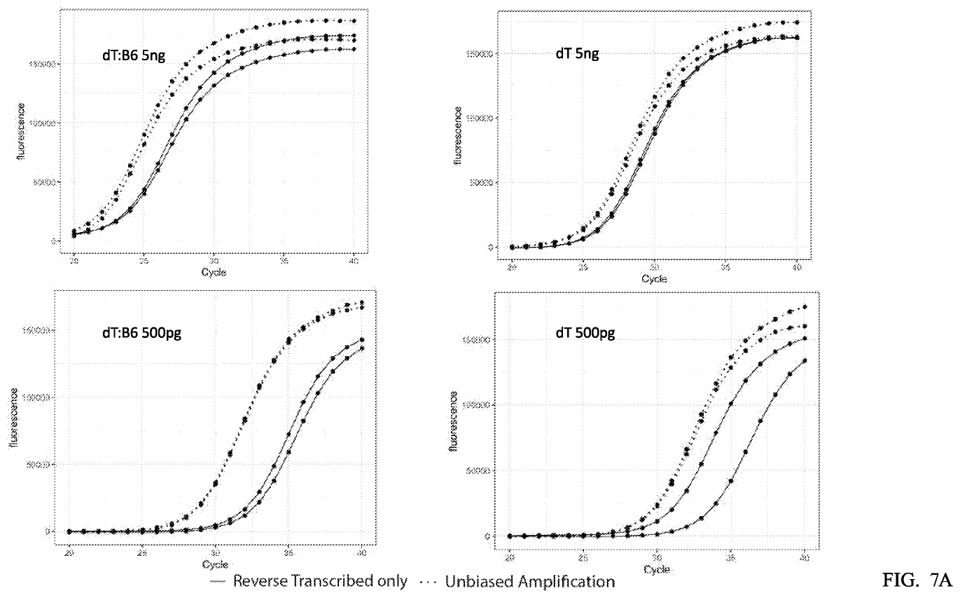

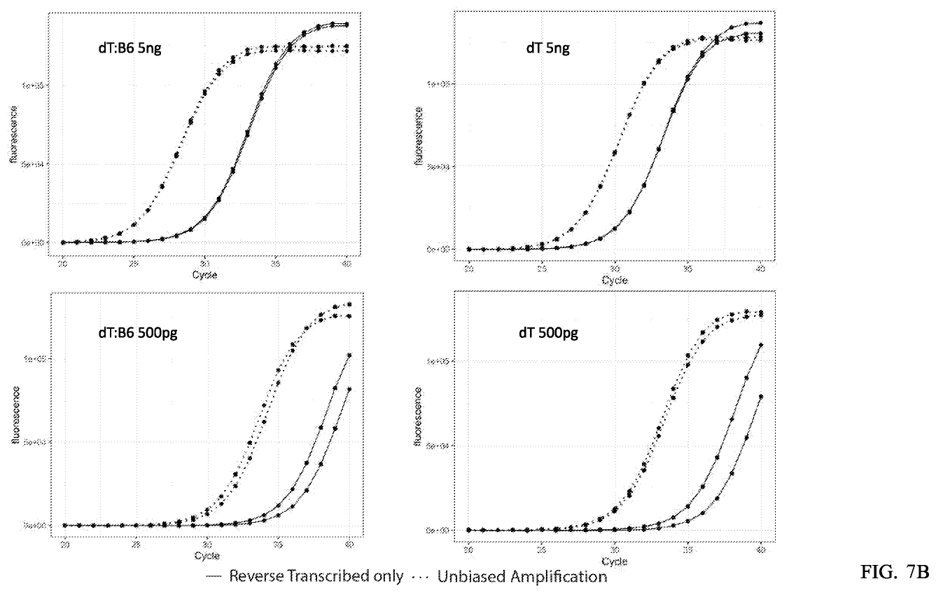

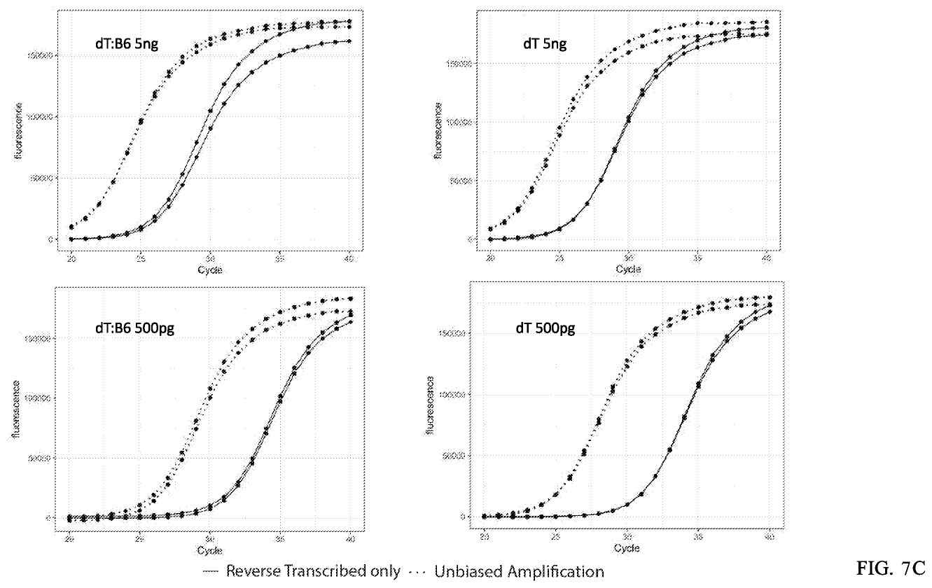

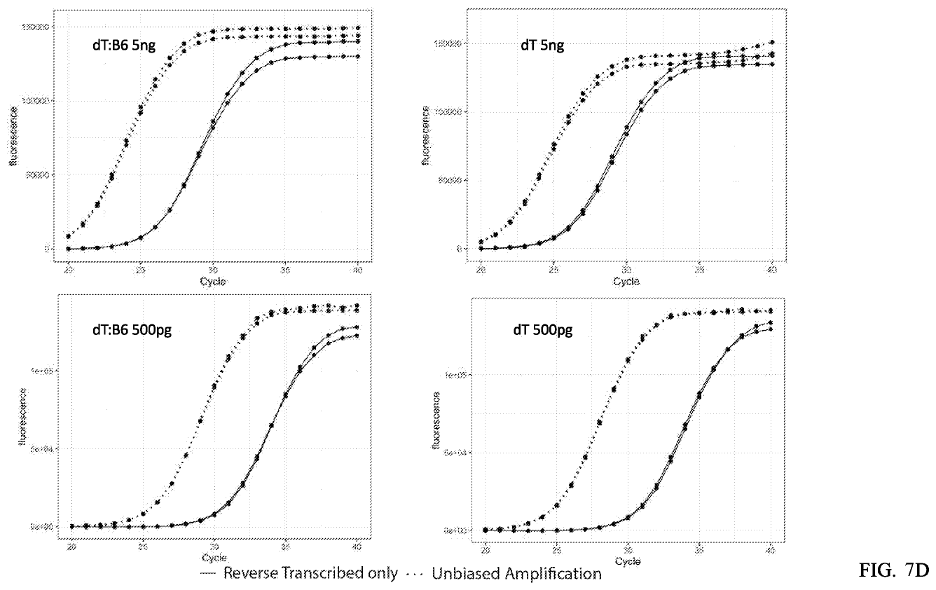

5 μl of RT is equivalent to 5 ng/500 pg of HEK293 RNA input and 10 μl of the Ampure XP cleaned library preparation is equivalent to the same input. The RT reaction mix saved from Example 1 was diluted 1:20 and the library prep was diluted 1:10, where 1 was used for each qPCR. qPCR was then performed using primers against human 18S (FIG. 7A), U2AF1 (FIG. 7B), GAPDH coding region (FIG. 7C) and GAPDH 3′UTR (FIG. 7D). After amplification according to the method described herein, all amplicons were demonstrated to be amplified using the unbiased amplification method described herein.

### Example 5—DNA Fragmentation and Library Preparation

3 μl of the Ampure XP purified library from Example 1 was then digested by MspJI in a 5 μl reaction as per manufacturer's instructions for 15 minutes at 37° C. to fragment the library. 1.5 μl of double stranded adaptors, one of them is represented by (5′-AGATGTGTATAAGAGACAG-3′) (SEQ ID NO: 6) and the other with 4 nt 5′ overhangs is represented by (5′-NNNNCTGTCTCTTATACACATCT-3′) (SEQ ID No: 7), 7 μl of 2× Quick Ligase Buffer (NEB), 0.5 μl 2 mM dNTPs, 0.3 μl Recombinant Taq and 0.7 μl of Quick ligase were then added to ligate adaptors to the ends. This was then amplified with two primers (5′-TCGTCGGCAGCGTCAGATGTGTATAAGAGACAG-3′) (SEQ ID NO: 8) and 5′-GTCTCGTGGGCTCGGAGATGTGTATAAGAGACAG-3′) (SEQ ID NO: 9) in a KOD reaction for 10 cycles with 60° C. annealing temperature with 40 second elongation. The reaction was then barcoded using ILLUMINA barcoding primers and sequenced on an iSeq 100 as per manufacturer's protocol.

The followings are examples providing thermocycler setting and conditions (in Tables 3-10) used to generate the library from the reagents according to Tables 1 and 2:
1) Add up to 3 μl of sample nucleic acid to 1 μl of primers represented by SEQ ID NOs:1-2, reaction mix of nucleotides and water and further top up to 4 μl with nuclease-free water if required. Put in thermocycler with the following setting (Table 1).

TABLE 1
Thermocycler settings

| Temperature (° C.) | Time (mins:secs) |
|---|---|
| 70° C. | 1:00 |
| 4° C. | hold |

2) Add 1 μl of a reverse transcriptase (from NEB) and put in thermocycler with the following setting (Table 2).

TABLE 2
Thermocycler settings

| Temperature (° C.) | Time (mins:secs) |
|---|---|
| 40° C. | 15:00 |
| 70° C. | 3:00 |
| 4° C. | hold |

3) Add 4 μl of primer represented by SEQ ID NO: 3 and a reaction mix including an isothermal buffer, oligonucleotides and water, and 1 μl of a polymerase with strand displacement activity on ice to bring the sample to a total of 10 μl and put in thermocycler with the following setting (Table 3).

TABLE 3
Thermocycler settings

| Temperature | Time |
|---|---|
| (° C.) | (mins:secs) |
| 70° C. | 0:10 |
| 4° C. | 0:10 |
| 45° C. | 1:00 |
| 48° C. | 1:00 |
| 51° C. | 1:00 |
| 54° C. | 1:00 |
| 57° C. | 1:00 |
| 60° C. | 10:00 |
| 25° C. | hold |

4) Add 15 μl of reagents from KOD kit and a pair of amplification primers represented by SEQ ID NOs: 4 and 5, respectively, wherein cytosine at 9^{th} position from 5′end of each of the amplification primers is methylated, and put in thermocycler with the following setting (Table 4).

TABLE 4
Thermocycler settings

| Temperature (° C.) | Time (mins:secs) | No. of cycles |
|---|---|---|
| 95° C. | 2:00 | |
| 95° C. | 0:08 | 15x |
| 58° C. | 0:10 | |
| 70° C. | 2:00 | |
| 25° C. | hold | |

5) Add 25 μl of Ampure XP (diluted) beads that has been brought up to room temperature and mix at least 10 times up and down with a pipette and let the sample incubate with the beads at room temperature for at least 3 minutes.
6) Use a magnetic rack to pull out the beads. Leave on rack for at least 2 minutes.
7) Remove supernatant, and resuspend the beads in 100 μl of freshly prepared 80% ethanol. Leave on magnetic rack for 20 seconds to pull out the beads. Repeat this step once more.
8) Remove supernatant and allow the beads to dry in room temperature for 5 minutes, then resuspend well in 8 μl of nuclease-free water. Incubate for 1 minute at room temperature before putting on the magnetic rack.
9) Carefully remove the eluate and transfer to a new tube.
10) Take 4 μl of the eluate and add 1 μl of reagents from MspJI kit in a new tube and put in thermocycler with the following setting (Table 5).

TABLE 5
Thermocycler settings

| Temperature (° C.) | Time (mins:secs) |
|---|---|
| 37° C. | 12:00 |
| 70° C. | 5:00 |
| 4° C. | Hold |

11) Add 10 μl of reagents from Quick Ligase and Taq kits with primers represented by SEQ ID NOs.: 6 and 7, respectively, to the sample to give a total of 15 μl and put in thermocycler with the following setting (Table 6).

TABLE 6
Thermocycler settings

| Temperature (° C.) | Time (mins:secs) |
|---|---|
| 25° C. | 15:00 |
| 70° C. | 5:00 |
| 25° C. | Hold |

12) Add 25 μl of reagents from KOD kit with primer represented by SEQ ID NOs.: 8-9, respectively, to the sample to give a total of 40 μl and put in thermocycler with the following setting (Table 7).

TABLE 7
Thermocycler settings

| Temperature (° C.) | Time (mins:secs) | No. of cycles |
|---|---|---|
| 95° C. | 2:00 | |
| 95° C. | 0:08 | 13x |
| 60° C. | 0:10 | |
| 70° C. | 0:40 | |
| 25° C. | hold | |

13) Take 5 μl of the sample from step 12, add 0.8 μl of each barcoding primer and 13.4 μl of the same reagents from KOD kit as in step 12 to give a total of 20 μl and put in thermocycler with the following setting (Table 8).

TABLE 8
Thermocycler settings

| Temperature (° C.) | Time (mins:secs) | No. of cycles |
|---|---|---|
| 95° C. | 2:00 | |
| 95° C. | 0:08 | 7x |
| 55° C. | 0:10 | |
| 70° C. | 0:40 | |
| 25° C. | hold | |

14) Add 20 μl of Ampure XP (diluted) beads that has been brought up to room temperature and mix at least 10 times up and down with a pipette and let the sample incubate with the beads at room temperature for at least 3 minutes.
15) Use a magnetic rack to pull out the beads. Leave on rack for at least 2 minutes.
16) Remove supernatant, and resuspend the beads in 100 μl of freshly prepared 80% ethanol. Leave on magnetic rack for 20 seconds to pull out the beads. Repeat this step once more.
17) Remove supernatant and allow the beads to dry in room temperature for 5 minutes, then resuspend well in 8 μl of nuclease-free water. Incubate for 1 minute at room temperature before putting on the magnetic rack.
18) Carefully remove the eluate and transfer to a new tube.
19) The library is ready for sequencing.
N.B.: If the box in the column showing the number of cycles is blank, it may stand for 1 cycle.

The results of the sequencing as shown in FIG. 8 and in Table 9 suggest that the library prepared according to the present method could detect over 9000 genes expressed in HEK cells with a read depth of between 60000-90000 reads.

TABLE 9

| | dT_B6_5ng | dT_5ng |
|---|---|---|
| Total Reads | 63858 | 84849 |
| Number of | 9410 | 12266 |
| transcripts mapped | | |

Furthermore, the read distributions of the dT_5 ng and dT_B6_5 ng samples are very similar, except the transcripts without a polyA tail, such as 7SK, mt-RNR1 and mt-RNR2. These results suggest that the present method generates an unbiased representative library.

### Example 6—Comparison Between N, B, D, H and NTC in Terms of Background Amplification

To identify potential background amplification, 0.4 μl of 5 μM of any one of N6 (CAGACTACCATGACCTGAGTCNNNNNN) (SEQ ID NO: 10), B6 (CAGACTACCATGACCTGAGTCBBBBBB) (SEQ ID NO: 1), D6 (CAGACTACCATGACCTGAGTCDDDDDD) (SEQ ID NO: 11) or H6 (CAGACTACCATGACCTGAGTCHHHHHH) (SEQ ID NO: 12) was mixed with 0.2 μl of 25 mM dNTPs, 1.0 μl of 5× PROMEGA MMLV reverse transcriptase buffer and 3.1 μl of water, heated to 70° C. for 30 seconds and cooled to 4° C. Then 0.5 μl of PROMEGA MMLV reverse transcriptase was added and the reactions incubated at 40° C. for 15 minutes, followed by 70° C. for 5 minutes.

1.5 μl NEB Isothermal Buffer, 0.75 μl 25 mM MgSO4, 1.5 μl 2 mM dNTP, 0.5 μl Bst3.0, 4.8 μl water and 1.0 μl of N8 (GTCAGAGTCGAATGCGTACTGNNNNNNNN) (SEQ ID NO: 13), B8 (GTCAGAGTCGAATGCGTACTGBBBBBBBB) (SEQ ID NO: 3), D8 (GTCAGAGTCGAATGCGTACTGDDDDDDDD) (SEQ ID NO: 14) or H6 (GTCAGAGTCGAATGCGTACTGHHHHHHHH) (SEQ ID NO: 15) was added to the respective sample tubes as described above.

The samples were incubated at 45° C. for 20s, increasing in steps of 1° C. every 20s until the samples reaches 60° C. The samples were further incubated at 60° C. for 10 mins.

The resultant sample are diluted 1/100 and 1 μl put in a 20 μl qPCR reaction using Solis Biodyne's FIREPOL SyBr Green qPCR mastermix as per manufacturer's instruction and the following primers (GTCAGAGTCGAATGCGTACTG) (SEQ ID NO: 16) and (CAGACTACCATGACCTGAGTC) (SEQ ID NO: 17), cycling with the following protocol: 95° C. 15s, 60° C. 15s, 72° C. 40s for 50 cycles.

The resultant qPCR Ct (Table 10) reveals the background level of an ‘empty’ reaction using different sets of random hexamers and octamers. With N, a low Ct indicates that background signal is significantly higher than with B, D or H set of bases.

Therefore, repeating B, D, or H of random hexamer or octamer at 3′ end in the adaptor sequence of the first and second DNA strand generation primers reduce the chance of non-specific priming over the repeating N in the same region of the strand generation primers.

TABLE 10

| Sample | N6 + N8 | B6 + B8 | D6 + D8 | H6 + H8 | NTC |
|---|---|---|---|---|---|
| Replicate 1 | 14.9 | 15.44 | 18.76 | 20.91 | 31.33 |
| Replicate 2 | 14.89 | 15.38 | 17.93 | 20.92 | 26.15 |

### Example 7

The following examples illustrates how DNA library is prepared according to other embodiments of the present invention:
1. To make 5 μM B5V1: dT, mix 9.5 μl of 100 μM B5V1 (represented by SEQ ID NO: 1) and 0.5 μl dT (represented by SEQ ID NO: 2) with 190 μl water;
2. To use 100 μM B7V1 (represented by SEQ ID NO: 3) as it comes in from the oligonucleotide manufacturer;
3. To mix 3 μl 100 μM mC F (represented by SEQ ID NO: 4, wherein the cytosine at the 9^{th} nucleotide from the 5′end is methylated), 3 μl 100 μM mC R (represented by SEQ ID NO: 5, wherein the cytosine at the 9^{th} nucleotide from the 5′end is methylated), 1 μl 10 mM dmCTP (NEB) and 23 μl water;
4. To mix 3 μl 100 μM Adaptor F (represented by SEQ ID NO: 6), 3 μl 100 μM Adaptor R (represented by SEQ ID NO: 7), 3 μl 1M sodium chloride, 3 μl 10×TE buffer, 18 μl water. Heat to 95° C. and cool down to 4° C. at 0.25° C. per second, to make 10 μm Adaptor mix;
5. To mix 15p 100 μM i5 F (represented by SEQ ID NO: 8), 15 μl 100 μM i7 R (represented by SEQ ID NO: 9) to make 50 μM i5 i7 Amp.

15 μl tubes are prepared with different primers ready for barcoding in a separate box.

### Example 8—Functional Quality Control (QC) of DNA Library Preparation

5 ng of HEK293 in 3 μl is processed with the AmpRE kit, then run on gel or TapeStation. 200-500 nt smear should be seen.

It will be appreciated by a person skilled in the art that other variations and/or modifications may be made to the embodiments disclosed herein without departing from the spirit or scope of the disclosure as broadly described. For example, in the description herein, features of different exemplary embodiments may be mixed, combined, interchanged, incorporated, adopted, modified, included etc. or the like across different exemplary embodiments. The present embodiments are, therefore, to be considered in all respects to be illustrative and not restrictive.

Unless otherwise defined, all terms of art, notations and other scientific terms or terminology used herein are intended to have the meanings commonly understood by those of skill in the art to which this invention pertains. In some cases, terms with commonly understood meanings are defined herein for clarity and/or for ready reference, and the inclusion of such definitions herein should not necessarily be construed to represent a substantial difference over what is generally understood in the art. It will be further understood that terms, such as those defined in commonly used dictionaries, should be interpreted as having a meaning that is consistent with their meaning in the context of the relevant art and/or as otherwise defined herein.

1. In a metagenomics sequencing process of a body fluid sample, the improvement comprising:
degrading unwanted nucleic acids in the sample by contacting the sample with magnetic particles coupled to enzymes to degrade unwanted nucleic acids present in the sample, wherein the magnetic particles coupled to enzymes are capable of degrading both DNA and RNA followed by applying a magnetic field to remove the magnetic particles coupled to enzymes from the sample; and
creating a DNA and RNA library from the degraded sample in a one-pot process.

2. The metagenomics sequencing process of claim 1, wherein the enzyme is a nuclease.

3. The metagenomics sequencing process of claim 1, wherein the one-pot process uses a set of primers to prime single-stranded DNA and at the same time prime RNA.

4. The metagenomics sequencing process of claim 1, wherein the sample includes more than one type of nucleic acid including single-stranded and/or double-stranded DNA and/or RNA as a template of subsequent extensions and amplifications and the one-pot process includes preparing a first DNA strand from the sample including annealing one or more first DNA strand generation primers to any of the DNA and/or RNA template, and extending from the annealed first DNA strand generation primer including employing a DNA polymerase that enables synthesis of the first DNA strand from either or both of DNA and/or RNA templates to obtain the first DNA strand.

5. The metagenomics sequencing process of claim 1, wherein the magnetic particles coupled to enzymes comprise magnetic particles coupled to both a DNAse and a RNAse, a magnetic particle coupled to an enzyme capable of degrading both DNA and RNA, a mixture comprising a magnetic particle coupled to a DNAse and a magnetic particle coupled to a RNAse, or combinations thereof.

6. The metagenomics sequencing process of claim 1, wherein the unwanted nucleic acids comprise cell-free nucleic acids.

7. The metagenomics sequencing process of claim 1, wherein the degrading unwanted nucleic acids in the sample further comprises applying a lysing agent to the sample for selectively lysing biological complexes in the sample to free said unwanted nucleic acids for degradation by the enzymes, optionally wherein the lysing agent is substantially incapable of lysing viruses and intact cells selected from bacteria, protozoa, fungi and parasites.

8. The metagenomics sequencing process of claim 6, wherein the lysing agent comprise a mild detergent.

9. The metagenomics sequencing process of claim 1, wherein the sample comprises a sample derived from a host subject and said unwanted nucleic acids comprise host nucleic acids but are substantially devoid of non-host nucleic acids.

10. The metagenomics sequencing process of claim 9, wherein the enzyme is a nuclease.

11. A method of detecting non-host species in a host sample, the method comprising:
contacting the host sample with magnetic particles coupled to enzymes to degrade cell-free host nucleic acids present in the host sample, wherein the magnetic particles coupled to enzymes are capable of degrading both DNA and RNA;
applying a magnetic field to remove the magnetic particles coupled to enzymes from the host sample;
creating a DNA and RNA library from the degraded sample in a one-pot process; and
detecting the presence of non-host nucleic acids from the DNA and RNA library.

12. The method of claim 11, wherein the one-pot process uses a set of primers to prime single-stranded DNA and at the same time prime RNA.

13. The method of claim 11, wherein the sample includes more than one type of nucleic acid including single-stranded and/or double-stranded DNA and/or RNA as a template of subsequent extensions and amplifications and the one-pot process includes preparing a first DNA strand from the sample including annealing one or more first DNA strand generation primers to any of the DNA and/or RNA template, and extending from the annealed first DNA strand generation primer including employing a DNA polymerase that enables synthesis of the first DNA strand from either or both of DNA and/or RNA templates to obtain the first DNA strand.

14. The method of claim 11, further comprising removing host cells from the host sample prior to contacting the host sample with magnetic particles coupled to enzymes.

15. The method of claim 11, further comprising applying a first lysing agent to the host sample for selectively lysing biological complexes to free host nucleic acids contained therein prior to the contacting step, optionally wherein the first lysing agent is substantially incapable of lysing non-host species to free the nucleic acids contained therein for degradation by the enzymes.

16. The method of claim 15, wherein the lysing agent comprise a mild detergent.

17. The method of claim 11, wherein the step of detecting the presence of non-host nucleic acids in the sample comprises:
applying a second lysing agent to the host sample for lysing non-host species to free the nucleic acids contained therein.

18. The method of claim 11, wherein the second lysing agent comprises lysing particles that are capable of imparting a mechanical force to lyse the non-host species and free the nucleic acids contained therein.

19. The method of claim 11, wherein detecting the presence of non-host nucleic acids in the sample comprises sequencing the non-host nucleic acids.
